SEQUENCE LISTING (82 sequences)

SEQ ID NO: 1 - DNA, 1102 bp, Artificial Sequence
  Note Artificial Sequence = Synthetic Construct
tacggatgagactgaaagaaaacctcaaaggagaactctgagaattcatctcaggatgaagctgcaagggcaaaggagaagtacttgtgacacaagcaaatgggtccattgatcaacagatgcaagaagattcttctcccaactactgtacctcctgcaacgatgagaatctggctccttggaggcctgctgccattcctgctgctcctctctggcctgcagagacccacagagggttctgaggttgcaattaaaatcgacttcgacttcgcaccaggttcttttgatgatcagtaccaaggctgtagcaaacaggttatggagaaactaactcaaggggattatttcacaaaagacatagaagcccagaagaattattttaggatgtggcaaaaagcccacttagcctggcttaaccaaggaaaagttctaccccagaacatgactaccacacacgctgtggctattttgttttacacattgaacagcaatgttcattctgactttactagagccatggcctctgttgccaggactccacagcagtatgaacgttcattccacttcaaatatttacactactacctcacctcagcaatccagctgctgaggaaagacagcatcatggagaatggcactctgtgctatgaggtgcattataggacgaaggatgtccactttaatgcctacacaggggccaccattcgatttggccaattcctttccacatccctcctgaaagaagaggcacaggagtttgggaaccagacactatttaccatattcacctgcctgggtgcacctgtacagtacttctccctcaagaaggaagtcttgatccctccctatgagctgtttaaagttataaatatgagctaccacccaagaggaaactggttgcagttgaggtcaactgggaacctgagcacatataactgtcagctgctaaaagcttccagcaagaaatgcatccctgatcctatagctattgcatctctctcctttttgaccagtgtcatcatcttttccaaaagcagagtataaagaaatcttgtggctccttttatttaaaaaaaatatttaaataaaatctttttttttg

SEQ ID NO: 2 - protein, 314 aa, Artificial Sequence
  Note Artificial Sequence = Synthetic Construct
MGPLINRCKKILLPTTVPPATMRIWLLGGLLPFLLLLSGLQRPTEGSEVAIKIDFDFAPGSFDDQYQGCSKQVMEKLTQGDYFTKDIEAQKNYFRMWQKAHLAWLNQGKVLPQNMTTTHAVAILFYTLNSNVHSDFTRAMASVARTPQQYERSFHFKYLHYYLTSAIQLLRKDSIMENGTLCYEVHYRTKDVHFNAYTGATIRFGQFLSTSLLKEEAQEFGNQTLFTIFTCLGAPVQYFSLKKEVLIPPYELFKVINMSYHPRGNWLQLRSTGNLSTYNCQLLKASSKKCIPDPIAIASLSFLTSVIIFSKSRV

SEQ ID NO: 3 - DNA, 1102 bp, Artificial Sequence
  Note Artificial Sequence = Synthetic Construct
tacggatgagactgaaagaaaacctcaaaggagaactctgagaattcatctcaggatgaagctgcaagggcaaaggagaagtacttgtgacacaagcaaatgggtccattgatcaacagatgcaagaagattcttctcccaactactgtacctcctgcaacgatgagaatctggctccttggaggcctgctgccattcctgctgctcctctctggcctgcagagacccacagagggttctgaggttgcaattaaaatcgacttcgacttcgcaccaggttcttttgatgatcagtaccaaggctgtagcaaacaggttatggagaaactaactcaaggggattatttcacaaaagacatagaagcccagaagaattattttaggatgtggcaaaaagcccacttagcctggcttaaccaaggaaaagttctaccccagaacatgactaccacacacgctgtggctattttgttttatacattgaacagcaatgttcattctgactttactagagccatggcctctgttgccaggactccacagcagtatgaacgttcattccacttcaaatatttacactactacctcacctcagcaatccagctgctgaggaaagacagcatcatggagaatggcactctgtgctatgaggtgcattataggacgaaggatgtccactttaatgcctacacaggggccaccattcgatttggccaattcctctccacatccctcctgaaagaagaggcacaggagtttgggaaccagacactatttaccatattcacctgcctgggtgcacctgtacagtacttctccctcaagaaggaagtcttgatccctccctatgagctgtttaaagttataaatatgagctaccacccaagaggagactggttgcagttgaggtcaactgggaacctgagcacatataactgtcagctgctaaaagcttccagcaagaaatgcatccctgatcctatagctattgcatctctctcctttttgaccagtgtcatcatcttttccaaaagcagagtataaagaaatcttgtggctccttttatttaaaaaaaatatttaaataaaatctttttttttg

SEQ ID NO: 4 - protein, 314 aa, Artificial Sequence
  Note Artificial Sequence = Synthetic Construct
MGPLINRCKKILLPTTVPPATMRIWLLGGLLPFLLLLSGLQRPTEGSEVAIKIDFDFAPGSFDDQYQGCSKQVMEKLTQGDYFTKDIEAQKNYFRMWQKAHLAWLNQGKVLPQNMTTTHAVAILFYTLNSNVHSDFTRAMASVARTPQQYERSFHFKYLHYYLTSAIQLLRKDSIMENGTLCYEVHYRTKDVHFNAYTGATIRFGQFLSTSLLKEEAQEFGNQTLFTIFTCLGAPVQYFSLKKEVLIPPYELFKVINMSYHPRGDWLQLRSTGNLSTYNCQLLKASSKKCIPDPIAIASLSFLTSVIIFSKSRV

SEQ ID NO: 5 - DNA, 30 bp, Artificial Sequence
  Note Artificial Sequence = Synthetic Construct
atgggtccattgatcaacagatgcaagaag

SEQ ID NO: 6 - DNA, 30 bp, Artificial Sequence
  Note Artificial Sequence = Synthetic Construct
attcttctcccaactactgtacctcctgca

SEQ ID NO: 7 - DNA, 30 bp, Artificial Sequence
  Note Artificial Sequence = Synthetic Construct
acgatgagaatctggctccttggaggcctg

SEQ ID NO: 8 - DNA, 30 bp, Artificial Sequence
  Note Artificial Sequence = Synthetic Construct
ctgccattcctgctgctcctctctggcctg

SEQ ID NO: 9 - DNA, 30 bp, Artificial Sequence
  Note Artificial Sequence = Synthetic Construct
cagagacccacagagggttctgaggttgca

SEQ ID NO: 10 - DNA, 30 bp, Artificial Sequence
  Note Artificial Sequence = Synthetic Construct
attaaaatcgacttcgacttcgcaccaggt

SEQ ID NO: 11 - DNA, 30 bp, Artificial Sequence
  Note Artificial Sequence = Synthetic Construct
tcttttgatgatcagtaccaaggctgtagc

SEQ ID NO: 12 - DNA, 30 bp, Artificial Sequence
  Note Artificial Sequence = Synthetic Construct
aaacaggttatggagaaactaactcaaggg

SEQ ID NO: 13 - DNA, 30 bp, Artificial Sequence
  Note Artificial Sequence = Synthetic Construct
gattatttcacaaaagacatagaagcccag

SEQ ID NO: 14 - DNA, 30 bp, Artificial Sequence
  Note Artificial Sequence = Synthetic Construct
aagaattattttaggatgtggcaaaaagcc

SEQ ID NO: 15 - DNA, 30 bp, Artificial Sequence
  Note Artificial Sequence = Synthetic Construct
cacttagcctggcttaaccaaggaaaagtt

SEQ ID NO: 16 - DNA, 30 bp, Artificial Sequence
  Note Artificial Sequence = Synthetic Construct
ctaccccagaacatgactaccacacacgct

SEQ ID NO: 17 - DNA, 30 bp, Artificial Sequence
  Note Artificial Sequence = Synthetic Construct
gtggctattttgttttacacattgaacagc

SEQ ID NO: 18 - DNA, 30 bp, Artificial Sequence
  Note Artificial Sequence = Synthetic Construct
aatgttcattctgactttactagagccatg

SEQ ID NO: 19 - DNA, 30 bp, Artificial Sequence
  Note Artificial Sequence = Synthetic Construct
gcctctgttgccaggactccacagcagtat

SEQ ID NO: 20 - DNA, 30 bp, Artificial Sequence
  Note Artificial Sequence = Synthetic Construct
gaacgttcattccacttcaaatatttacac

SEQ ID NO: 21 - DNA, 30 bp, Artificial Sequence
  Note Artificial Sequence = Synthetic Construct
tactacctcacctcagcaatccagctgctg

SEQ ID NO: 22 - DNA, 30 bp, Artificial Sequence
  Note Artificial Sequence = Synthetic Construct
aggaaagacagcatcatggagaatggcact

SEQ ID NO: 23 - DNA, 30 bp, Artificial Sequence
  Note Artificial Sequence = Synthetic Construct
ctgtgctatgaggtgcattataggacgaag

SEQ ID NO: 24 - DNA, 30 bp, Artificial Sequence
  Note Artificial Sequence = Synthetic Construct
gatgtccactttaatgcctacacaggggcc

SEQ ID NO: 25 - DNA, 30 bp, Artificial Sequence
  Note Artificial Sequence = Synthetic Construct
accattcgatttggccaattcctttccaca

SEQ ID NO: 26 - DNA, 30 bp, Artificial Sequence
  Note Artificial Sequence = Synthetic Construct
tccctcctgaaagaagaggcacaggagttt

SEQ ID NO: 27 - DNA, 30 bp, Artificial Sequence
  Note Artificial Sequence = Synthetic Construct
gggaaccagacactatttaccatattcacc

SEQ ID NO: 28 - DNA, 30 bp, Artificial Sequence
  Note Artificial Sequence = Synthetic Construct
tgcctgggtgcacctgtacagtacttctcc

SEQ ID NO: 29 - DNA, 30 bp, Artificial Sequence
  Note Artificial Sequence = Synthetic Construct
ctcaagaaggaagtcttgatccctccctat

SEQ ID NO: 30 - DNA, 30 bp, Artificial Sequence
  Note Artificial Sequence = Synthetic Construct
gagctgtttaaagttataaatatgagctac

SEQ ID NO: 31 - DNA, 30 bp, Artificial Sequence
  Note Artificial Sequence = Synthetic Construct
cacccaagaggaaactggttgcagttgagg

SEQ ID NO: 32 - DNA, 30 bp, Artificial Sequence
  Note Artificial Sequence = Synthetic Construct
tcaactgggaacctgagcacatataactgt

SEQ ID NO: 33 - DNA, 30 bp, Artificial Sequence
  Note Artificial Sequence = Synthetic Construct
cagctgctaaaagcttccagcaagaaatgc

SEQ ID NO: 34 - DNA, 30 bp, Artificial Sequence
  Note Artificial Sequence = Synthetic Construct
atccctgatcctatagctattgcatctctc

SEQ ID NO: 35 - DNA, 30 bp, Artificial Sequence
  Note Artificial Sequence = Synthetic Construct
tcctttttgaccagtgtcatcatcttttcc

SEQ ID NO: 36 - DNA, 15 bp, Artificial Sequence
  Note Artificial Sequence = Synthetic Construct
aaaagcagagtataa

SEQ ID NO: 37 - DNA, 30 bp, Artificial Sequence; identical to SEQ ID NO: 5
  Note Artificial Sequence = Synthetic Construct

SEQ ID NO: 38 - DNA, 30 bp, Artificial Sequence; identical to SEQ ID NO: 6
  Note Artificial Sequence = Synthetic Construct

SEQ ID NO: 39 - DNA, 30 bp, Artificial Sequence; identical to SEQ ID NO: 7
  Note Artificial Sequence = Synthetic Construct

SEQ ID NO: 40 - DNA, 30 bp, Artificial Sequence; identical to SEQ ID NO: 8
  Note Artificial Sequence = Synthetic Construct

SEQ ID NO: 41 - DNA, 30 bp, Artificial Sequence; identical to SEQ ID NO: 9
  Note Artificial Sequence = Synthetic Construct

SEQ ID NO: 42 - DNA, 30 bp, Artificial Sequence; identical to SEQ ID NO: 10
  Note Artificial Sequence = Synthetic Construct

SEQ ID NO: 43 - DNA, 30 bp, Artificial Sequence; identical to SEQ ID NO: 11
  Note Artificial Sequence = Synthetic Construct

SEQ ID NO: 44 - DNA, 30 bp, Artificial Sequence; identical to SEQ ID NO: 12
  Note Artificial Sequence = Synthetic Construct

SEQ ID NO: 45 - DNA, 30 bp, Artificial Sequence; identical to SEQ ID NO: 13
  Note Artificial Sequence = Synthetic Construct

SEQ ID NO: 46 - DNA, 30 bp, Artificial Sequence; identical to SEQ ID NO: 14
  Note Artificial Sequence = Synthetic Construct

SEQ ID NO: 47 - DNA, 30 bp, Artificial Sequence; identical to SEQ ID NO: 15
  Note Artificial Sequence = Synthetic Construct

SEQ ID NO: 48 - DNA, 30 bp, Artificial Sequence; identical to SEQ ID NO: 16
  Note Artificial Sequence = Synthetic Construct

SEQ ID NO: 49 - DNA, 30 bp, Artificial Sequence
  Note Artificial Sequence = Synthetic Construct
gtggctattttgttttatacattgaacagc

SEQ ID NO: 50 - DNA, 30 bp, Artificial Sequence; identical to SEQ ID NO: 18
  Note Artificial Sequence = Synthetic Construct

SEQ ID NO: 51 - DNA, 30 bp, Artificial Sequence; identical to SEQ ID NO: 19
  Note Artificial Sequence = Synthetic Construct

SEQ ID NO: 52 - DNA, 30 bp, Artificial Sequence; identical to SEQ ID NO: 20
  Note Artificial Sequence = Synthetic Construct

SEQ ID NO: 53 - DNA, 30 bp, Artificial Sequence; identical to SEQ ID NO: 21
  Note Artificial Sequence = Synthetic Construct

SEQ ID NO: 54 - DNA, 30 bp, Artificial Sequence; identical to SEQ ID NO: 22
  Note Artificial Sequence = Synthetic Construct

SEQ ID NO: 55 - DNA, 30 bp, Artificial Sequence; identical to SEQ ID NO: 23
  Note Artificial Sequence = Synthetic Construct

SEQ ID NO: 56 - DNA, 30 bp, Artificial Sequence; identical to SEQ ID NO: 24
  Note Artificial Sequence = Synthetic Construct

SEQ ID NO: 57 - DNA, 30 bp, Artificial Sequence
  Note Artificial Sequence = Synthetic Construct
accattcgatttggccaattcctctccaca

SEQ ID NO: 58 - DNA, 30 bp, Artificial Sequence; identical to SEQ ID NO: 26
  Note Artificial Sequence = Synthetic Construct

SEQ ID NO: 59 - DNA, 30 bp, Artificial Sequence; identical to SEQ ID NO: 27
  Note Artificial Sequence = Synthetic Construct

SEQ ID NO: 60 - DNA, 30 bp, Artificial Sequence; identical to SEQ ID NO: 28
  Note Artificial Sequence = Synthetic Construct

SEQ ID NO: 61 - DNA, 30 bp, Artificial Sequence; identical to SEQ ID NO: 29
  Note Artificial Sequence = Synthetic Construct

SEQ ID NO: 62 - DNA, 30 bp, Artificial Sequence; identical to SEQ ID NO: 30
  Note Artificial Sequence = Synthetic Construct

SEQ ID NO: 63 - DNA, 30 bp, Artificial Sequence
  Note Artificial Sequence = Synthetic Construct
cacccaagaggagactggttgcagttgagg

SEQ ID NO: 64 - DNA, 30 bp, Artificial Sequence; identical to SEQ ID NO: 32
  Note Artificial Sequence = Synthetic Construct

SEQ ID NO: 65 - DNA, 30 bp, Artificial Sequence; identical to SEQ ID NO: 33
  Note Artificial Sequence = Synthetic Construct

SEQ ID NO: 66 - DNA, 30 bp, Artificial Sequence; identical to SEQ ID NO: 34
  Note Artificial Sequence = Synthetic Construct

SEQ ID NO: 67 - DNA, 30 bp, Artificial Sequence; identical to SEQ ID NO: 35
  Note Artificial Sequence = Synthetic Construct

SEQ ID NO: 68 - DNA, 15 bp, Artificial Sequence
  Note Artificial Sequence = Synthetic Construct
aaaagcagagtataa

SEQ ID NO: 69 - DNA, 21 bp, Artificial Sequence
  Note Artificial Sequence = Synthetic Construct
ccattcctgctgctcctctct

SEQ ID NO: 70 - DNA, 25 bp, Artificial Sequence
  Note Artificial Sequence = Synthetic Construct
tctggggtagaacttttccttggtt

SEQ ID NO: 71 - DNA, 24 bp, Artificial Sequence
  Note Artificial Sequence = Synthetic Construct
ggctctgcggagaccaggagtcag

SEQ ID NO: 72 - DNA, 25 bp, Artificial Sequence
  Note Artificial Sequence = Synthetic Construct
ttaccagacatggctccgcttggaa

SEQ ID NO: 73 - DNA, 25 bp, Artificial Sequence
  Note Artificial Sequence = Synthetic Construct
gaccgcgctttgcttgctgagtttg

SEQ ID NO: 74 - DNA, 25 bp, Artificial Sequence
  Note Artificial Sequence = Synthetic Construct
gctggggtggtgaacactgtgctga

SEQ ID NO: 75 - DNA, 28 bp, Artificial Sequence
  Note Artificial Sequence = Synthetic Construct
atgggtccattgatcaacagatgcaaga

SEQ ID NO: 76 - DNA, 29 bp, Artificial Sequence
  Note Artificial Sequence = Synthetic Construct
ttatactctgcttttggaaaagatgatga

SEQ ID NO: 77 - DNA, 28 bp, Artificial Sequence
  Note Artificial Sequence = Synthetic Construct
ccatcttcttcaaggacgacggcaacta

SEQ ID NO: 78 - DNA, 23 bp, Artificial Sequence
  Note Artificial Sequence = Synthetic Construct
gggcggactgggtgctcaggtag

SEQ ID NO: 79 - DNA, 21 bp, Artificial Sequence
  Note Artificial Sequence = Synthetic Construct
gctcgtcgtcgacaacggctc

SEQ ID NO: 80 - DNA, 25 bp, Artificial Sequence
  Note Artificial Sequence = Synthetic Construct
caaacatgatctgggtcatcttctc

SEQ ID NO: 81 - DNA, 16 bp, Artificial Sequence
  Note Artificial Sequence = Synthetic Construct
gtccacccgaagcttg

SEQ ID NO: 82 - DNA, 16 bp, Artificial Sequence
  Note Artificial Sequence = Synthetic Construct
cntgaagttctcagga

(19) United States
(12) Patent Application Publication
(10) Pub. No.: US 2005/0100896 A1
(43) Pub. Date: May 12, 2005

(54) Identification of the dombrock blood group glycoprotein as a polymorphic member of the adp-ribosyltransferase gene family

(76) Inventors: Jeffery L. Miller, Potomac, MD (US); Alexander Gubin, North Potomac, MD (US); Marion E. Reid, Jamaica Estates, NY (US)

Correspondence Address: NATIONAL INSTITUTE OF HEALTH;C/O NEEDLE & ROSENBERG, P.C., SUITE 1000, 999 PEACHTREE STREET, ATLANTA, GA, 30303, US

(21) Appl. No.: 10/381,325

(22) PCT Filed: Sep. 24, 2001
(86) PCT No.: PCT/US01/29954

Related U.S. Application Data
(60) Provisional application No. 60/235,162, filed on Sep. 23, 2000.

(51) Int. Cl. C12Q 1/68; C07H 21/04; C12N 9/00; A61K 39/00; C12N 15/74

(52) U.S. Cl. 435/6; 435/69.1; 435/252.3; 435/183; 435/320.1; 424/185.1; 536/23.2

(57) ABSTRACT

The present invention provides an isolated peptide comprising a Dombrock carrier molecule that contains either a Dombrock (a) antigen or a Dombrock (b) antigen. Further, the present invention provides a nucleic acid encoding a Dombruck carrier molecule or fragments thereof. Moreover, a nucleic acid encoding a Dombrock (a) antigen or fragments thereof is provided. The present invention also provides a nucleic acid encoding a Dombrock (b) antigen or fragments thereof. Kits comprising antibodies to either the Dombrock (a) antigen or the Dombrock (b) antigen are also provided. Moreover, kits comprising either the Dombrock (a) antigen or the Dombrock (b) antigen, and kits comprising nucleic acids that encode the Dombrock (a) antigen or the Dombrock (b) antigen are provided. Methods for detecting the Dombrock (a) antigen or the Dombrock (b) antigen, and methods for detecting antibodies specifically binding to the Dombrock (a) antigen or the Dombrock (b) antigen are provided.

This application claims the benefit of priority of U.S. Provisional Application No. 60/235,162, filed Sep. 23, 2000, which is incorporated by reference herein in its entirety.

### BACKGROUND OF THE INVENTION

1. Field of the Invention

The present invention relates to the field of transfusion medicine and preventing in subjects hemolytic complications associated with the transfusion of mismatched blood types. Specifically, the present invention relates to a method of identifying a red blood cell antigen on a carrier molecule that can cause severe hemolytic reactions in a recipient subject, if the donor and recipient are not properly cross-matched.

2. Background Art

Based upon their clinical importance for transfusion medicine, 25 genetically distinct molecules on human erythrocytes collectively known as the human blood groups¹ have been extensively studied. Over 200 antigenic variations of these molecules have been defined by serology and genetic linkage studies. Most of the molecules have been cloned and identified as a functionally diverse group of membrane transporters, complement regulatory molecules, adhesion molecules, and ectoenzymes². Molecular genetic studies have strongly linked blood group polymorphisms to the severity of malarial disease^{3, 4}. In addition, polymorphic loci associated with blood antigenicity may be useful for linkage studies for a variety of other diseases⁵. Hence, molecular and functional aspects of human blood groups provide a rich source of information relevant for studies of erythroid cell biology, human polymorphisms, and transfusion medicine.

The Dombrock blood group was discovered with serological tests of blood and named after the original Do^{a} serum donor in 1965⁶. Nearly a decade passed before the discovery of the antithetical antigen (Do^{b})⁷. These antigens are quite informative as genetic markers with Do^{a} gene frequencies of 0.42, 0.33, 0.12, and 0.07 in Northern European, Black Americans, Japanese, and Thai populations, respectively⁸. Three additional antigens (Gy^{a}, Hy, and Jo^{a}) carried on the Dombrock blood group molecule are “high incidence” antigens with gene frequencies predicted at greater than 99% in all populations studied⁹. Clinically, the blood group has not been associated with hemolytic disease of the newborn, but severe hemolytic transfusion reactions due to the presence of anti-Dombrock antibodies have been reported among adults^{10,11}. Detection of Dombrock-mediated hemolysis is difficult, even though the clinical relevance of detecting anti-Dombrock has recently been emphasized for sickle-cell disease patients receiving multiple blood transfusions¹².

Despite over 30 years of antigen-based studies of the Dombrock blood group system, the identity of the Dombrock carrier molecule itself has not been determined. The present invention identifies the gene, DOK1, and its characterization as the Dombrock carrier molecule.

### SUMMARY OF THE INVENTION

The present invention provides an isolated peptide having the amino acid sequence of SEQ ID NO: 2, wherein the peptide is a Dombrock (a) antigen.

The present invention also provides an isolated peptide having the amino acid sequence of SEQ ID NO:4, wherein the peptide is a Dombrock (b) antigen.

The present invention provides a kit comprising red blood cells having a Dombrock (a) antigen.

The present invention also provides a kit comprising red blood cells having a Dombrock (b) antigen.

Further provided by the present invention is a method of detecting in a sample an antibody directed to a Dombrock carrier molecule, comprising a) contacting red blood cells having a Dombrock carrier molecule with the sample and b) detecting agglutination of the red blood cells, whereby agglutination of the red blood cells indicates the presence in the sample of an antibody to a Dombrock carrier molecule.

Also provided is a method of detecting in a sample an antibody directed to a Dombrock carrier molecule, comprising a) contacting a purified Dombrock carrier molecule with the sample and b) detecting an antigen/antibody complex, whereby detection of the antigen/antibody complex indicates the presence of the antibody directed to the antigen.

The present invention provides a method of detecting a Dombrock carrier molecule on red blood cells, comprising a) contacting the red blood cells with an antibody directed to the Dombrock carrier molecule and b) detecting agglutination of the red blood cells, whereby agglutination of the red blood cells indicates the presence of the Dombrock carrier molecule.

Also provided by the present invention is a method of detecting in a sample a Dombrock carrier molecule, comprising a) contacting the sample with an antibody directed to a Dombrock carrier molecule and b) detecting a carrier molecule/antibody complex, whereby detection of the carrier molecule/antibody complex indicates the presence of the Dombrock carrier molecule in the sample.

The present invention provides a method of detecting in a sample, a cell that expresses a Dombrock (a) antigen, comprising detecting in the sample a nucleic acid that encodes a Dombrock (a) antigen.

The present invention also provides a method of detecting in a sample, a cell that expresses a Dombrock (b) antigen, comprising detecting in the sample a nucleic acid that encodes a Dombrock (b) antigen.

The present invention provides a method of detecting a subject having a Dombrock carrier molecule, comprising a) performing amplification of a nucleic acid of the subject by contacting a nucleic acid from a cell of the subject with a primer, consisting of nucleotides from a nucleic acid encoding a Dombrock carrier molecule and b) detecting an amplification product from step (a), whereby the detection of an amplification product identifies the subject as having a Dombrock carrier molecule.

### BRIEF DESCRIPTION OF THE DRAWINGS

FIG. 1 shows FISH detection of DOK1. Metaphase spreads from synchronized human peripheral lymphocytes were hybridized with digoxigenin-11-dUTP-labeled DOK1 probe. Hybridization signals were detected with rhodamine-conjugated anti-digoxigenin antibodies. Normal metaphase spread after FISH (left panel) shows localization of DOK1 on chromosome 12 (arrows). Inverted DAPI banding (right panel) simulates G-banding for chromosomal identification. Inset displays subchromosomal localization of the gene on the short arm 12 p12.3-13.1 of chromosome 12.

FIG. 2 shows molecular organization of DOK1. FIG. 2A is a schematic representation of DOK1 gene. Exons and introns are depicted as hatched and black bars, respectively. Numbers show exon/intron boundries relative to position of DOK1 start codon at position 1. Open boxes show signal peptides, (•) indicate putative N-linked glycosylation sites. (•) indicate putative N-myristoylation sites. FIG. 2B shows the predicted amino acid sequence of DOK1. Signal peptide, the predicted signal peptide; GPI-anchor motif, the predicted GPI anchor; ART depicts the ADP-ribosyltransfrease motif. (•) indicate amino acid position corresponding to identified single nucleotide polymorphisms (SNPs).

FIG. 3 shows expression of DOK1 in CD34⁺, Epo-stimulated cells. CD34⁺ cells were grown in the presence of 1U/ml Epo, and total RNA was purified from the cells collected on respective days 0-12. The RT-PCR products (top panel-DOK1, middle panel-PBGD, lower panel-CD34) from each day were separated in 1.2% agarose gel. (M) 100 bp DNA Ladder, (N) no DNA control.

FIG. 4 shows a northern blot analysis of DOK1 expression in human hematologic tissues. Lanes 1-6 contain, in order, 2 μg polyA⁺ RNA from human spleen, lymph node, thymus, peripheral blood leukocytes, bone marrow, fetal liver. ³²P labeled DOK1 probe was used for hybridization. RNA size marker bands are indicated in the left margin of the blot.

FIG. 5 shows a northern blot of pI2E versus pDI2E transfected K562. Each lane contains 10 μg of total RNA from untransfected K562 (lane 1), cells transfected with pI2E vector (lane 2), and cells transfected with pDI2E (lane 3). ³²P-labeled probes were used for hybridizations. A. Membrane hybridized with DOK1 probe. B. Membrane hybridized with EGFP probe. C. Membrane hybridized with β-actin probe.

FIG. 6 shows flow cytometric analyses of pI2E (DOK1 negative) versus pDI2E (DOK1 positive) transfected K562 cells. K562 cells were transfected with pI2E or pDI2E plasmid DNA and grown in G418 containing media. Stable transfectants were stained with serum containing antibodies to Dombrock antigens (Gy^{a}, Do^{a}, Jo^{a}, Hy), counterstained with PE labeled F(ab′)₂ fragments and analyzed by flow cytometry. The numbers shown at the upper right of each panel are the mean fluorescence values (PE channel) of the transfected cells. The control serum contained no Dombrock antibodies as measured by indirect hemagglutination assays.

FIG. 7 shows glycosylphosphatidylinositol anchoring of DOK1. FIG. 7A shows K562 cells that were transfected with pDI2E construct and stable transfectants were selected in G418 containing media. The cells were then incubated in PI-PLC (+) or buffer (−) prior to analysis. Flow cytometry was performed using FITC-conjugated anti-CD71, anti-CD59, and Dombrock antisera for comparison. FIG. 7B shows K562 cells and GPI-negative cell line transfected either with pI2E or pDI2E plasmid DNA and selected in G418 containing media were stained with Dombrock antisera and analyzed by flow cytometry. The mean fluorescence of each population is shown at the top of each panel.

### DETAILED DESCRIPTION OF THE INVENTION

As used herein, “a,” “an” and “the” may mean one or more than one. For example, “a” peptide may mean one peptide or more than one peptide. Moreover, “the” peptide may mean one peptide or more than one peptide.

The present invention provides an isolated peptide having the amino acid sequence of SEQ ID NO:2, wherein the peptide is a Dombrock (a) antigen. A “peptide” is an amino acid sequence of at least 5 amino acids, including polypeptides and proteins. “Isolated” as used herein means the peptide, polypeptide or nucleic acid of this invention is sufficiently free of contaminants or cell components with which peptides, polypeptides or nucleic acids normally occur and is present in such concentration as to be the only significant peptide, polypeptide or nucleic acid present in the sample. “Isolated” does not mean that the preparation must be technically pure (homogeneous), but it is sufficiently pure to provide the peptide, polypeptide or nucleic acid in a form in which it can be used therapeutically or diagnostically. A “Dombrock (a) antigen” is found in nature on a Dombrock carrier molecule which is located on a red blood cell membrane.

The invention also provides a fragment of the Dombrock carrier molecule that is specific for the Dombrock carrier molecule and may or may not contain a region that is specific for either either Dombrock (a) antigen or Dombrock (b) antigen. This may elicit a response by an existing antibody rather than producing an antibody.

Also provided by the present invention is an isolated antigenic fragment of the peptide identified as SEQ ID NO:2, wherein the fragment comprises the amino acid asparagine at position 265. This fragment is specific for SEQ ID NO:2.

“Antigenic” when used herein to describe any of the fragments of the invention means capable of binding specifically to an antibody. A “fragment” as used herein means a molecule of at least 5 contiguous amino acids of a particular peptide that has at least one function shared by the peptide or a region thereof, for example, antigenicity. It is contemplated that fragments of at least 10, 20, 30, 40, 50, 60, 70, 80, 90, 100, 150, 200, 250, 300 or 310 amino acids are within the scope of the present invention. It is also contemplated that fragments of up to 10 amino acids are within the scope of the present invention. As used herein to describe an amino acid sequence (protein, polypeptide, peptide, etc.), “specific” means that the amino acid sequence is not found identically in any other source. The determination of specificity is made routine, because of the availability of computerized amino acid sequence databases, wherein an amino acid sequence of almost any length can be quickly and reliably checked for the existence of identical sequences. If an identical sequence is not found, the protein is “specific” for the recited source.

An antigenic fragment of at least about 5 consecutive amino acids of the peptide identified as in SEQ ID NO:2 is provided and binds an antibody. An antigenic fragment can be selected by applying the routine technique of epitope mapping to the peptide identified as SEQ ID NO:2 to determine the regions of the proteins and fragments that contain epitopes reactive with serum antibodies or are capable of eliciting an immune response in an animal. Once the epitope is selected, an antigenic polypeptide containing the epitope can be synthesized directly, or produced recombinantly by cloning nucleic acids encoding the polypeptide in an expression system, according to the standard methods. Alternatively, an antigenic fragment of the antigen can be isolated from the whole antigen or a larger fragment by chemical or mechanical disruption. Fragments can also be randomly chosen from the amino acid sequence identified as SEQ ID NO:2 and synthesized. The purified fragments thus obtained can be tested to determine their antigenicity and specificity by routine methods. Specific examples of fragments of the Dombrock carrier molecule or the Dombrock (a) antigen are those that consist of or comprise the amino acid sequences encoded by the nucleic acids identified in SEQ ID NOS:5-36.

The present invention provides an isolated nucleic acid encoding the peptide identified as SEQ ID NO:2. An example of a nucleic acid encoding the peptide of SEQ ID NO:2 is the nucleic acid comprising the nucleotide sequence of SEQ ID NO: 1. Further, the present invention provides an isolated nucleic acid fragment comprising at least 10 contiguous nucleotides of a nucleic acid encoding the polypeptide of SEQ ID NO:2, for example, the nucleotide sequence of SEQ ID NO:1. The nucleic acid fragment may encode an antigenic fragment of SEQ ID NO:2. Moreover, any Dombrock-specific fragment of SEQ ID NO: 1 is useful, not just those that encode antigenic peptides. Examples of fragments of SEQ ID NO: 1 include, but are not limited to, nucleic acids SEQ ID NOS:5-36. In fact, both shorter and longer nucleic acids that contain at least 10 contiguous nucleotides of SEQ ID NOS:5-36 can be identified as specific for Dombrock encoding nucleic acids. Further, SEQ ID NO:17 comprises a single nucleotide polymorphism of the gene encoding the Dombrock carrier molecule at position 18; SEQ ID NO:25 comprises a single nucleotide polymorphism of the gene encoding the Dombrock carrier molecule at position 24; and SEQ ID NO:31 comprises a single nucleotide polymorphism of the gene encoding the Dombrock carrier molecule at position 13. These single nucleotide polymorphisms (SNPs) make these nucleic acid fragments and nucleic acids that include them specific for subjects with Dombrock (a) antigen. Nucleic acids specific for the gene and cDNA encoding the Dombrock carrier molecule are provided. The accession number for the human DOK1 sequence in GenBank is AF290204. Fragments of the nucleic acid of SEQ ID NO: 1 may encode fragments of the Dombrock carrier molecule or fragments of the amino acid sequence of SEQ ID NO:2 that contain the amino acid that determines the Dombrock (a) antigen.

The present invention provides an isolated peptide having the amino acid sequence of SEQ ID NO:4, wherein the peptide is a Dombrock (b) antigen. A “Dombrock (b) antigen” is found in nature on a Dombrock carrier molecule which is located on a red blood cell membrane.

Also provided by the present invention is an isolated antigenic fragment of the peptide identified as SEQ ID NO:4, wherein the fragment comprises the amino acid aspartic acid at position 265.

An antigenic fragment of at least about 5 consecutive amino acids of the peptide identified as in SEQ ID NO:4 is provided and binds an antibody. An antigenic fragment can be selected by applying the routine technique of epitope mapping to the peptide identified as SEQ ID NO:4 to determine the regions of the proteins and fragments that contain epitopes reactive with serum antibodies or are capable of eliciting an immune response in an animal. Once the epitope is selected, an antigenic polypeptide containing the epitope can be synthesized directly, or produced recombinantly by cloning nucleic acids encoding the polypeptide in an expression system, according to the standard methods. Alternatively, an antigenic fragment of the antigen can be isolated from the whole antigen or a larger fragment by chemical or mechanical disruption. Fragments can also be randomly chosen from the amino acid sequence identified as SEQ ID NO:4 and synthesized. The purified fragments thus obtained can be tested to determine their antigenicity and specificity by routine methods. Specific examples of fragments of the Dombrock carrier molecule or the Dombrock (b) antigen are those that consist of or comprise the amino acid sequences encoded by the nucleic acids identified in SEQ ID NOS:37-68.

Modifications to any of the above proteins or fragments of the present invention can be made, while preserving the specificity and activity (function) of the native protein or fragment thereof. As used herein, “native” describes a protein that occurs in nature. The modifications contemplated herein can be conservative amino acid substitutions, for example, the substitution of a basic amino acid for a different basic amino acid. Modifications can also include creation of fusion proteins with epitope tags or known recombinant proteins or genes encoding them created by subcloning into commercial or non-commercial vectors (e.g., polyhistidine tags, flag tags, myc tag, glutathione-S-transferase [GST] fusion protein, xylE fusion reporter construct). Furthermore, the modifications contemplated will not affect the function of the protein or the way the protein accomplishes that function (e.g., its secondary structure or the ultimate result of the protein's activity. The means for determining these parameters are well known.

The present invention provides an isolated nucleic acid encoding the peptide identified as SEQ ID NO:4. An example of a nucleic acid encoding the peptide of SEQ ID NO:4 is the nucleic acid comprising the nucleotide sequence of SEQ ID NO:3. Further, the present invention provides an isolated nucleic acid fragment comprising at least 10 contiguous nucleotides of a nucleic acid encoding the polypeptide of SEQ ID NO:4, for example, the nucleotide sequence of SEQ ID NO:3. The nucleic acid fragment may encode an antigenic fragment of SEQ ID NO:4. Moreover, any Dombrock-specific fragment of SEQ ID NO:3 is useful, not just those that encode antigenic peptides. Examples of fragments of SEQ ID NO:3 include, but are not limited to, nucleic acids SEQ ID Nos:37-68. In fact, both shorter and longer nucleic acids that contain at least 10 contiguous nucleotides of SEQ ID NOS:37-68 can be identified as specific for Dombrock encoding nucleic acids. Further, SEQ ID NO:49 comprises a single nucleotide polymorphism of the gene encoding the Dombrock carrier molecule at position 18; SEQ ID NO:57 comprises a single nucleotide polymorphism of the gene encoding the Dombrock carrier molecule at position 24; and SEQ ID NO:63 comprises a single nucleotide polymorphism of the gene encoding the Dombrock carrier molecule at position 13. These single nucleotide polymorphisms (SNPs) make these nucleic acid fragments and nucleic acids that include them specific for subjects with Dombrock (b) antigen. Also, nucleic acids specific for the gene and cDNA encoding the Dombrock carrier molecule are provided. Fragments of the nucleic acid of SEQ ID NO:3 may encode fragments of the Dombrock carrier molecule or fragments of the amino acid sequence of SEQ ID NO:4 that contain the amino acid that determines the Dombrock (b) antigen.

“Nucleic acid” as used herein includes single- or double-stranded molecules which may be DNA, comprised of the nucleotide bases A, T, C and G, or RNA, comprised of the bases A, U (substitutes for T), C, and G. The nucleic acid may represent a coding strand or its complement. Nucleic acids may be identical in sequence to the portion of the sequence which is naturally occurring or may include alternative codons which encode the same amino acid as that which is found in the naturally occurring sequence. Furthermore, nucleic acids can include codons which represent conservative substitutions of amino acids as are well known in the art. Nucleic acids can have marker molecules, labels, etc.

For example, the present invention provides an isolated fragment of the nucleic acid identified as SEQ ID NO:1, comprising at least 10 nucleotides, wherein the fragment comprises cytosine at position 477 of SEQ ID NO: 1.

In another example, the present invention provides an isolated fragment of the nucleic acid identified as SEQ ID NO:1, comprising at least 10 nucleotides, wherein the fragment comprises thymine at position 723 of SEQ ID NO: 1.

A further example of an isolated fragment of the nucleic acid identified as SEQ ID NO:1 comprises at least 10 nucleotides, wherein the fragment comprises adenine at position 892 of SEQ ID NO:1.

For example, the present invention provides an isolated fragment of the nucleic acid identified as SEQ ID NO:3, comprising at least 10 nucleotides, wherein the fragment comprises thymine at position 477 of SEQ ID NO:3.

In another example, the present invention provides an isolated fragment of the nucleic acid identified as SEQ ID NO:3, comprising at least 10 nucleotides, wherein the fragment comprises cytosine at position 723 of SEQ ID NO:3.

A further example of an isolated fragment of the nucleic acid identified as SEQ ID NO:3 comprises at least 10 nucleotides, wherein the fragment comprises guanine at position 892 of SEQ ID NO:3.

An isolated nucleic acid of at least 10 nucleotides that specifically hybridizes with the nucleic acid of either SEQ ID NO: 1 or a fragment thereof, or the nucleic acid of SEQ ID NO:3 or a fragment thereof, under selected conditions is provided. For example, the conditions can be polymerase chain reaction conditions and the hybridizing nucleic acid can be a primer consisting of a specific fragment of the reference sequence or a nearly identical nucleic acid that hybridizes only to the exemplified nucleic acid sequences, for example SEQ ID NO: 1 and SEQ ID NO:3.

The invention provides an isolated nucleic acid that specifically hybridizes with the gene encoding the Dombrock carrier molecule shown in the sequence set forth as SEQ ID Nos: 1 and 3 under the conditions of about 16 hrs at about 65° C., about 5×SSC, about 0.1% SDS, about 2× Denhardt's solution, about 150 μg/ml salmon sperm DNA with washing at about 65° C., 30 min, 2×, in about 0.1×SSPE/0.1% SDS. Alternative hybridization conditions include 68° C. for about 16 hours in buffer containing about 6×SSC, 0.5% sodium dodecyl sulfate, about 5× Denhardt's solution and about 100 μg salmon sperm DNA, with washing at about 60° C. in about 0.5×SSC. For example, the hybridizing nucleic acid can be a probe that hybridizes only to the exemplified Dombrock carrier molecule gene or a homolog thereof. Thus, the hybridizing nucleic acid can be a naturally occurring homolog of the exemplified Dombrock carrier molecule genes. The hybridizing nucleic acid can also include insubstantial base substitutions that do not prevent hybridization under the stated conditions or affect the function of the encoded protein, the way the protein accomplishes that function (e.g., its secondary structure or the ultimate result of the protein's activity). The means for determining these parameters are well known.

As used herein to describe nucleic acids, the term “selectively hybridizes” excludes the occasional randomly hybridizing nucleic acids as well as nucleic acids that encode other known homologs of the present proteins. The selectively hybridizing nucleic acids of the invention can have at least 70%, 73%, 78%, 80%, 85%, 88%, 90%, 91%, 92%, 93%, 94%, 95%, 96%, 97%, 98% and 99% complementarity with the segment and strand of the sequence to which it hybridizes. The nucleic acids can be at least 10, 18, 20, 25, 50, 100, 150, 200, 300, 500, 550, 750, 900 nucleotides in length, depending on whether the nucleic acid is to be used as a primer, probe or for protein expression. Thus, the nucleic acid can be an alternative coding sequence for the protein, or can be used as a probe or primer for detecting the presence of the nucleic acid encoding a Dombrock carrier molecule. If used as primers, the invention provides compositions including at least two nucleic acids which selectively hybridize with different regions so as to amplify a desired region. Depending on the length of the probe or primer, it can range between 70% complementary bases and full complementarity and still hybridize under stringent conditions. The invention provides examples of these nucleic acids, so that the degree of complementarity required to distinguish selectively hybridizing from nonselectively hybridizing nucleic acids under stringent conditions can be clearly determined for each nucleic acid. It should also be clear that the hybridizing nucleic acids of the invention will not hybridize with nucleic acids encoding unrelated proteins (hybridization is selective) under stringent conditions.

“Stringent conditions” refers to the washing conditions used in a hybridization protocol. In general, the washing conditions should be a combination of temperature and salt concentration chosen so that the denaturation temperature is approximately 5-20° C. below the calculated Tₘ of the hybrid under study. The temperature and salt conditions are readily determined empirically in preliminary experiments in which samples of reference DNA immobilized on filters are hybridized to the probe or protein coding nucleic acid of interest and then washed under conditions of different stringencies. Examples of nucleic acids that may be used as primers and probes include, but are not limited to, the nucleic acid sequences of SEQ ID Nos: 5-68.

The present invention provides an antibody that specifically binds the Dombrock carrier molecule. Moreover, the invention provides an antibody that specifically binds to a fragment of the Dombrock carrier molecule.

The present invention provides an antibody that specifically binds the peptide identified as SEQ ID NO:2.

The present invention provides an antibody that specifically binds an antigenic fragment of the peptide identified as SEQ ID NO:2, wherein the fragment comprises asparagine at position 265 of SEQ ID NO:2. The antibodies of the present invention can be polyclonal or monoclonal.

The present invention provides an antibody that specifically binds the peptide identified as SEQ ID NO:4. The present invention provides an antibody that specifically binds an antigenic fragment of the peptide identified as SEQ ID NO:4, wherein the fragment comprises aspartic acid at position 265 of SEQ ID NO:4. The antibodies of the present invention can be polyclonal or monoclonal.

An antibody can specifically bind a unique epitope of the antigen. The term “bind” means the well understood antigen/antibody binding as well as other nonrandom association with an antigen. “Specifically bind” as used herein describes an antibody or other ligand that does not cross react substantially with any antigen other than the one specified, in this case, the peptide identified as SEQ ID NO:2. Antibodies can be made as described in Harlow and Lane. Briefly, the purified peptide or a fragment thereof can be injected into an animal in an amount and in intervals sufficient to elicit an immune response. The antibody can also be generated by delivering to the animal a nucleic acid (e.g., in a plasmid or other vector) that encodes the Dombrock carrier molecule or the Dombrock (a) antigen or the Dombrock (b) antigen under conditions whereby some antigen or carrier molecule is expressed in the animal. Polyclonal antibodies can be purified directly, or spleen cells from the animal can be fused with an immortal cell line and screened for monoclonal antibody secretion. Thus, purified monospecific polyclonal antibodies that specifically bind the antigen are within the scope of the present invention. The antibody can be monoclonal as is known to a person of skill in the art. Thus, a person of skill in the art can use a purified Dombrock (a) antigen to make a Dombrock (a)-specific antibody and a purified Dombrock (b) antigen to make a Dombrock (b)-specific antibody.

The present invention also provides a kit for detecting anti-Dombrock (a) antibodies, comprising an isolated antigenic peptide having the amino acid sequence of SEQ ID NO:2. or an isolated antigenic fragment of the peptide having the amino acid sequence of SEQ ID NO:2, wherein the fragment comprises asparagine at position 265 of SEQ ID NO:2.

The present invention also provides a kit for detecting anti-Dombrock (b) antibodies, comprising an isolated antigenic peptide having the amino acid sequence of SEQ ID NO:4. or an isolated antigenic fragment of the peptide having the amino acid sequence of SEQ ID NO:4, wherein the fragment comprises aspartic acid at position 265 of SEQ ID NO:4.

Also, included in antibody detection kits are the usual components of such kits. For example, the kit may also contain the antigens in a modified Alsever's Solution, neomycin, chloramphenicol and inosine to preserve carbohydrate metabolism. Other antibody potentiators known to a person of skill in the art may be present in the kits.

Also provided by the present invention is a kit for detecting a Dombrock (a) antigen comprising a monoclonal antibody that specifically binds to the peptide having the amino acid sequence of SEQ ID NO:2. Moreover, the present invention provides a kit for detecting a Dombrock (a) antigen comprising a monoclonal antibody that specifically binds an antigenic fragment of the peptide having the amino acid sequence of SEQ ID NO:2, wherein the fragment comprises asparagine at position 265 of SEQ ID NO:2.

Also provided by the present invention is a kit for detecting a Dombrock (b) antigen comprising a monoclonal antibody that specifically binds to the peptide having the amino acid sequence of SEQ ID NO:4. Moreover, the present invention provides a kit for detecting a Dombrock (b) antigen comprising a monoclonal antibody that specifically binds an antigenic fragment of the peptide having the amino acid sequence of SEQ ID NO:4, wherein the fragment comprises aspartic acid at position 265 of SEQ ID NO:4.

Also, included in antigen detection kits are the usual components of such kits. For example, the kit may also contain the antibodies in a modified Alsever's Solution, neomycin, chloramphenicol and inosine to preserve carbohydrate metabolism. Other antibody potentiators known to a person of skill in the art may be present in the kits.

The present invention further provides a kit for detecting a Dombrock (a) antigen comprising an isolated nucleic acid, encoding the peptide having an amino acid sequence of SEQ ID NO:2. Further provided by the present invention is a kit for detecting a Dombrock (a) antigen comprising an isolated nucleic acid, wherein the nucleic acid comprises the nucleotide sequence of SEQ ID NO: 1.

The present invention provides a kit for detecting a Dombrock (a) antigen, comprising an isolated fragment of the nucleotide sequence of SEQ ID NO:1, comprising at least 10 nucleotides, wherein the fragment comprises cytosine at position 477 of SEQ ID NO: 1.

The present invention provides a kit for detecting a Dombrock (a) antigen, comprising an isolated fragment of the nucleotide sequence of SEQ ID NO:1, comprising at least 10 nucleotides, wherein the fragment comprises thymine at position 723 of SEQ ID NO: 1.

The present invention provides a kit for detecting a Dombrock (a) antigen, comprising an isolated fragment of the nucleotide sequence of SEQ ID NO: 1, comprising at least 10 nucleotides, wherein the fragment comprises adenine at position 892 of SEQ ID NO:1.

Also, included in the kits for detecting a Dombrock (a) antigen, in addition to the nucleotides, are the usual components of such kits. For example, the kit may also contain deoxynucleoside triphosphates, DNA polymerase and standard buffers for PCR amplification.

The present invention further provides a kit for detecting a Dombrock (b) antigen comprising an isolated nucleic acid, encoding the peptide having an amino acid sequence of SEQ ID NO:4. Further provided by the present invention is a kit for detecting a Dombrock (b) antigen comprising an isolated nucleic acid, wherein the nucleic acid comprises the nucleotide sequence of SEQ ID NO:3.

The present invention provides a kit for detecting a Dombrock (b) antigen, comprising an isolated fragment of the nucleotide sequence of SEQ ID NO:3, comprising at least 10 nucleotides, wherein the fragment comprises thymine at position 477 of SEQ ID NO:3.

The present invention provides a kit for detecting a Dombrock (b) antigen, comprising an isolated fragment of the nucleotide sequence of SEQ ID NO:3, comprising at least 10 nucleotides, wherein the fragment comprises cytosine at position 723 of SEQ ID NO:3.

The present invention provides a kit for detecting a Dombrock (b) antigen, comprising an isolated fragment of the nucleotide sequence of SEQ ID NO:3, comprising at least 10 nucleotides, wherein the fragment comprises guanine at position 892 of SEQ ID NO:3.

Also, included in the kits for detecting a Dombrock (b) antigen, in addition to the nucleotides, are the usual components of such kits. For example, the kit may also contain deoxynucleoside triphosphates, DNA polymerase and standard buffers for PCR amplification.

Also provided by the present invention are kits comprising a microchip array comprising one or more of the following nucleic acids: a) an isolated nucleic acid encoding the peptide having an amino acid sequence of SEQ ID NO:2; b) the nucleic acid comprising the nucleotide sequence of SEQ ID NO: 1; c) an isolated fragment of a nucleic acid that encodes the peptide having an amino acid sequence of SEQ ID NO:2; d) an isolated fragment of the nucleotide sequence of SEQ ID NO:1, comprising at least nucleotides, wherein the fragment comprises cytosine at position 477 of SEQ ID NO:1; e) an isolated fragment of the nucleotide sequence of SEQ ID NO:1, comprising at least 10 nucleotides, wherein the fragment comprises thymine at position 723 of SEQ ID NO: 1; i) an isolated fragment of the nucleotide sequence of SEQ ID NO: 1, comprising at least 10 nucleotides, wherein the fragment comprises adenine at position 892 of SEQ ID NO:1; g) an isolated nucleic acid encoding the peptide having an amino acid sequence of SEQ ID NO:4; h) the nucleic acid comprising the nucleotide sequence of SEQ ID NO:3; i) an isolated fragment of a nucleic acid that encodes the peptide having an amino acid sequence of SEQ ID NO:4; j) an isolated fragment of the nucleotide sequence of SEQ ID NO:3, comprising at least 10 nucleotides, wherein the fragment comprises thymine at position 477 of SEQ ID NO:3; k) an isolated fragment of the nucleotide sequence of SEQ ID NO:3, comprising at least 10 nucleotides, wherein the fragment comprises cytosine at position 723 of SEQ ID NO:3; and 1) an isolated fragment of the nucleotide sequence of SEQ ID NO:3, comprising at least 10 nucleotides, wherein the fragment comprises guanine at position 892 of SEQ ID NO:3. A kit may comprise one or more nucleic acids that encode the Dombrock carrier molecule, the Dombrock (a) antigen, or the Dombrock (b) antigen or a combination of two or three of these molecules.

The present invention also provides a kit comprising red blood cells having a Dombrock (a) antigen. Moreover, a kit comprising red blood cells having a Dombrock (b) antigen is provided.

The present invention provides a method of detecting in a sample an antibody directed to a Dombrock carrier molecule, comprising a) contacting red blood cells having a Dombrock carrier molecule with the sample; and b) detecting agglutination of the red blood cells, whereby agglutination of the red blood cells indicates the presence in the sample of an antibody to a Dombrock carrier molecule. As used herein a “Dombrock carrier molecule” is a red blood cell membrane protein that comprises the peptides having either the amino acid sequence of SEQ ID NO:2 or the amino acid sequence of SEQ ID NO:4. Thus, the method of the present invention can detect an antibody directed to a Dombrock (a) antigen that has the amino acid sequence of SEQ ID NO:2. Moreover, the method of the present invention can detect an antibody directed to a Dombrock (b) antigen that has the amino acid sequence of SEQ ID NO:4. A “sample” can be from any biological source and includes, but is not limited to, body fluids such as blood, plasma, serum, lymph, saliva, urine, cerebrospinal fluid, semen, aqueous humor, vitreous and gastrointestinal secretions. Therefore, a person of skill in the art can contact red blood cells having a known Dombrock antigen, (a) or (b), with a sample and look for agglutination of red blood cells. The presence of agglutination indicates that an, antibody directed to either Dombrock (a) or Dombrock (b) is present in the sample. As used herein “agglutination” means the aggregation into clumps or masses of red blood cells upon exposure to a specific antibody.

The present invention also provides a method of detecting in a sample an antibody directed to a Dombrock carrier molecule, comprising a) contacting a purified Dombrock carrier molecule with the sample and b) detecting an antigen/antibody complex, whereby detection of the antigen/antibody complex indicates the presence of the antibody that specifically binds to the antigen. The conditions whereby an antigen/antibody complex can form and be detected can be standard conditions well known in the art for protocols such as immunoprecipitation, agglutination, Western blotting, etc. Examples of protocols for producing and detecting antigen/antibody complexes are provided in the Examples section herein. Thus, the method of the present invention can detect an antibody that specifically binds to a Dombrock (a) antigen, wherein the antigen has the amino acid sequence of SEQ ID NO:2. Moreover, the method of the present invention can detect an antibody that specifically binds to a Dombrock (b) antigen, wherein the antigen has the amino acid sequence of SEQ ID NO:4.

The present invention provides a method of detecting a Dombrock carrier molecule on red blood cells, comprising a) contacting the red blood cells with an antibody directed to the Dombrock carrier molecule and b) detecting agglutination of the red blood cells, whereby agglutination of the red blood cells indicates the presence of the Dombrock carrier molecule. The method can detect a Dombrock carrier molecule comprising a Dombrock (a) antigen when the antibody used is directed to and specifically binds the Dombrock (a) antigen. Similarly, the method of the present invention can detect a Dombrock carrier molecule comprising a Dombrock (b) antigen when the antibody used is directed to and specifically binds a Dombrock (b) antigen.

The present invention also provides a method of detecting in a sample a Dombrock carrier molecule, comprising a) contacting the sample with an antibody directed to a Dombrock carrier molecule and b) detecting a carrier molecule/antibody complex, whereby detection of the carrier molecule/antibody complex indicates the presence of the Dombrock carrier molecule in the sample. The antibody used in this method preferably specifically binds to the Dombrock carrier molecule. The method of the present invention can detect a Dombrock carrier molecule comprising a Dombrock (a) antigen when the antibody used is directed to and specifically binds a Dombrock (a) antigen. Similarly, the method of the present invention can detect a Dombrock carrier molecule comprising a Dombrock (b) antigen when the antibody used is directed to and specifically binds a Dombrock (b) antigen.

The present invention also provides a method of detecting a subject having a Dombrock carrier molecule, comprising a) performing amplification of a nucleic acid of the subject by contacting a nucleic acid from a cell of the subject with a primer that specifically hybridizes with a nucleic acid encoding a Dombrock carrier molecule under PCR conditions or high stringency probing conditions; and b) detecting an amplification product from step (a), whereby the detection of an amplification product identifies the subject as having a Dombrock carrier molecule. A person skilled in the art can choose primers consisting of nucleotides from a nucleic acid encoding a Dombrock carrier molecule, or fragment thereof, and detect an amplification product that identifies the subject as having a Dombrock carrier molecule. Any Dombrock-specific nucleic acid can be used to design a primer for specific amplification of a Dombrock carrier-encoding nucleic acid. Examples of primers used in a PCR protocol are given in the Examples below and in the attached sequence listing. Thus, the method of the present invention can detect a subject having a Dombrock carrier molecule comprising a Dombrock (a) antigen or a Dombrock (b) antigen.

Also provided by the present invention is a method of detecting in a sample, a cell that expresses a Dombrock (a) antigen, comprising detecting in the sample a nucleic acid that encodes a Dombrock (a) antigen. For example, a person of skill in the art can detect a Dombrock (a) antigen by a) contacting a nucleic acid from the sample with a probe that specifically hybridizes to a nucleic acid encoding a Dombrock (a) antigen and b) detecting the probe hybridized to the nucleic acid, whereby the presence of the hybridization indicates the presence of a cell that expresses a Dombrock (a) antigen. Additionally, PCR can be used, and more specifically, RT-PCR can be used to identify a cell that expresses an mRNA that encodes a Dombrock (a) antigen.

The present invention provides a method of detecting in a sample, a cell that expresses a Dombrock (b) antigen, comprising detecting in the sample a nucleic acid that encodes a Dombrock (b) antigen. For example, a person of skill in the art can detect a Dombrock (b) antigen by a) contacting a nucleic acid from the sample with a probe that specifically hybridizes to a nucleic acid encoding a Dombrock (b) antigen and b) detecting the probe hybridized to the nucleic acid, whereby the presence of the hybridization indicates the presence of a cell that expresses a Dombrock (b) antigen. Additionally, PC-R can be used, and more specifically, RT-PCR can be used to identify a cell that expresses an mRNA that encodes a Dombrock (b) antigen.

It is contemplated that the present invention provides a method of detecting in a sample, a cell that expresses a Dombrock carrier molecule, comprising detecting in the sample a nucleic acid that encodes a Dombrock (a) antigen or a Dombrock (b) antigen, wherein the detecting step comprises a) contacting a nucleic acid from the sample with a microchip array comprising a probe that specifically hybridizes a nucleic acid encoding a Dombrock (a) antigen or a Dombrock (b) antigen and b) detecting a signal generated by the nucleic acid hybridizing with the probe, whereby the presence of the signal indicates the presence of a nucleic acid that encodes either a Dombrock (a) antigen or a Dombrock (b) antigen. A person of skill in the art can use a kit for detection of the nucleic acids as described, for example, by Affymetrix® Corporation.

The following examples are put forth so as to provide those of ordinary skill in the art with a complete disclosure and description of how the compositions and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. The present invention is more particularly described in the following examples which are intended as illustrative only since numerous modifications and variations therein will be apparent to those skilled in the art.

### EXAMPLES

Cells and Cell Culture

The parental cell lines used in this study were obtained from ATCC, Rockville, Md. All primary cells were obtained from normal blood donors. Culture methods and flow cytometric sorting of the erythroid precursor cells has been described elsewhere¹⁶.

Library Construction and Sequencing

A cDNA library from human erythroid precursor cells was constructed using SMART™ PCR cDNA Library Construction Kit (Clontech, Palo Alto, Calif.) according to the manufacturer's directions with slight modifications. Briefly, reverse transcription was performed in the presence of 1 μM PNA oligos (N-terminal)-biotin-GTC-CAC-CCG-AAG-CTT-G-(C-terminal) and (N-terminal)-biotin-C(T/C)T-GAA-GTT-CTC-AGG-A-(C-terminal) designed to specifically suppress reverse transcription of globin transcripts. Synthesized cDNA was digested with SfiI and size-selected on a 1% agarose gel. cDNA fragments smaller than 800 bp were discarded. Large-scale sequencing of the library was done by the NIH Intramural Sequencing Center. Sequencing analyses revealed less than 2% of the ESTs shared homology with globin gene transcripts. More extensive sequencing of the DOK transcripts was performed in the laboratory using dRhodamine Terminator Cycle Sequencing Kit (PE Applied Biosystems, Foster City, Calif.). Sequence assemblies and analyses were performed using Sequencher™3.1 software (Gene Codes Corporation, Ann Arbor, Mich.). Single nucleotide polymorphisms (SNPs) were analyzed using Gene Inspector™ (Textco, West Lebanon, N.H.) software package.

Fluorescent In Situ Hybridization (FISH)

Metaphase spreads derived from 5-bromo-deoxyuridine synchronized peripheral lymphocytes of a normal male were used as a template. The DNA probe containing DOK1 was labeled with digoxigenin 11-dUTP by nick-translation and hybridization signals were detected with rhodamine-conjugated anti-digoxigenin antibodies (Roche Molecular Biochemicals, Indianapolis, Ind.). The condition of hybridization, detection of hybridization signals, digital-image acquisition, processing, and analysis were performed as previously described¹⁷. Chromosomes were identified by converting DAPI-banding into G-simulated banding using the IP Lab Image Software (Scanalytics, Fairfax, Va.). 15 metaphases were analyzed.

Reverse Transcriptase-Polymerase Chain Reaction (RT-PCR)

Total RNA was purified from cells using TRIzol® reagent (Life Technologies, Rockville, Md.) according to the supplied protocol. Reverse transcription (RT) was performed using oligo-(dT) primer and Superscipt™ Reverse Transcriptase (Life Technologies, Rockville, Md.) as suggested by the manufacturer at 42° C. for 50 minutes. One microliter of RT reaction was used in 50 μl PCR amplification with appropriate primers as follows: initial denaturation at 95° C. for 1 min, denaturing at 95° C. for 15 sec, annealing at 68° C. for 15 sec, extension at 72° C. for 20 sec for 28 cycles. PCR products were resolved in 1.2% agarose gel and stained with ethidium bromide. For DOK1 amplification (SEQ ID NO:69) 5′-CCATTCCTGCTGCTCCTCTCT-3′ and (SEQ ID NO:70) 5′-TCTGGGGTAGAACTTTTCCTTGGT-T-3′ primers were used (247 bp expected product size). For human housekeeping porphobilinogen deaminase (PBGD) amplification (SEQ ID NO:71) 5′-GGCTCTGCGGAGACCAGGAGTCAG-3′ and (SEQ ID NO:72) 5′-TTACCAGACATGGCTCCGCTTGGA A-3′ primers were used (expected product size is 153 bp). For CD34 amplification (SEQ ID NO:73) 5′-GACCGCGCTTTGCTTGCTGAGTTTG-3′ and (SEQ ID NO:74) 5′-GCTGGGGTGGTGAACACTGTGCTGA-3′ primers were used (expected product size is 324 bp).

Northern Blotting

Total RNA from K562 cells, K562 cells transformed with p12E versus pDI2E was isolated using TRIzol® reagent. Ten micrograms of RNA were electrophoresed in 1% agarose denaturing gel and transferred to Hybond® (Amersham Pharmacia Biotech, Piscataway, N.J.) nylon membrane using downward transfer method¹⁸ in 1× transfer buffer (Ambion, Austin, Tex.). RNA was crosslinked to the membrane using LTV Stratalinker 1800 (Stratagene, La Jolla, Calif.). Membrane was prehybridized in ULTRAhyb hybridization buffer (Ambion, Austin, Tex.) at 42° C. for 30 min. Buffer was replaced with the fresh ULTRAhyb containing ³²P labeled probe and hybridized at 42° C. for 16 hours. Membrane was washed in low and high stringency buffers (Ambion) according to the manufacturer's protocol and subjected to autoradiography. Probes for Northern blotting were generated by PCR, purified by QIAquick PCR Purification Kit (Qiagen, Valencia, Calif.) and labeled using DNA Labeling Beads (-dCTP) (Amersham Pharmacia Biotech, Piscataway, N.J.). Human Immune System Multiple Tissue Northern (MTN™) Blot U was processed as recommended by the manufacturer (Clontech, Palo Alto, Calif.).

Plasmids and Probes

The plasmid pDOKIIRES2-EGFP (pDI2E) was constructed by transferring EcoR1/SalI fragment containing the entire ORF of DOK1 into similarly digested pIRES2-EGFP (pI2E) vector (Clontech, Palo Alto, Calif.). The EcoR1/SalI fragment originated from the candidate DOK1 clone selected by screening of cDNA library constructed from human erythroid precursor cells using SMART™ PCR cDNA Library Construction Kit (Clontech, Palo Alto, Calif.). The library was converted into a plasmid form as suggested by the manufacturer where all inserts are unidirectionally cloned into SfiI sites of pTriplEx2 vector. The following primers were used to generate PCR products used as probes: (SEQ ID NO:75) 5′-ATGGGTCCATTGATCAACAGATGCAAGA-3′ and (SEQ ID NO:76) 5′-TTATACTCTGCTTTTGGAAAAGATGATGA-3′ for DOK1 (945 bp product, amplified from reverse transcribed HEL total RNA), (SEQ ID NO:77) 5′-CCATCTTCTTCAAGGACGACGGCAACTA-3′ and (SEQ ID NO:78) 5′-GGGCGGACTGGGTGCTCAGGTAG-3′ for EGFP (330 bp product, pEGFP-NI vector was a template). Primers (SEQ ID NO:79) 5′-GCTCGTCGTCGACAACGGCTC-3′ and (SEQ ID NO:80) 5′-CAAACATGATCTGGGTCATCTTCTC-3′ primers were used to generate the beta-actin probe (probe length is 353 bp). The BAC clone (GenBank accession AC007655) was obtained from Roswell Park Cancer Institute. The presence of the DOK1 in BAC AC007655 DNA was confirmed by PCR.

Flow Cytometric Analysis

K562 cells were transfected either with pI2E or pDI2E and grown for 21 days in G418 supplemented media (Biofliuds, Gaithersburg, Md.). GFP expressing cells were sorted and expanded for 4 weeks in G418 media. Untransfected K562 cells and cells transfected with pI2E or pDI2E were prepared for flow cytometry using a two-step protocol with the clinical sera samples. Prior to staining, each serum was diluted 1:10 with cold PBS and filtered through 0.45μ filter. 50 μl of the diluted serum was used to stain 10⁵ cells transfected with pI2E or pDI2E plasmids. The cells were incubated at 4° C. for 45 minutes, washed with cold Phosphate-Buffered Saline (PBS), and stained with goat anti-human IgG Phycoerythrin (PE) F(ab′)₂ fragment (Sigma, St Loius, Mo.) at a final dilution of 1:50. After 30 minutes incubation at 4° C., the cells were washed twice in PBS. Appropriate negative controls with the second step F(ab′)₂ fragment or serum alone were done in both untransfected cells and transfected pools.

Analyses were performed with Coulter Epics Elite flow cytometer (Beckman-Coulter, Hileah, Fla.) using a 488 nm argon laser. GFP was detected at 520-530 nm, and PE at 555-595 nm. Gated live cells expressing GFP were analyzed for DOK1 expression. Positive DOK1 cell populations were defined as those having a mean PE fluorescence at levels greater than two standard deviations above the negative controls. At least 2000 gated events were collected for each analysis.

PI-PLC Treatment

Release of GPI-anchored proteins from the surfaces of intact cells was attained by using phosphatidylinositol phospholipase C (PI-PLC) from Bacillus thuringiensis (Oxford Glycosciences, Wakefield, Mass.) according to manufacturer's directions. The cells were incubated in 200 μl of TBS (10 mM TRIS, pH 7.5, 150 mM NaCl) containing 2 U/ml of enzyme for 1 hour at 37° C. and washed before analysis.

Identification of a Candidate Gene

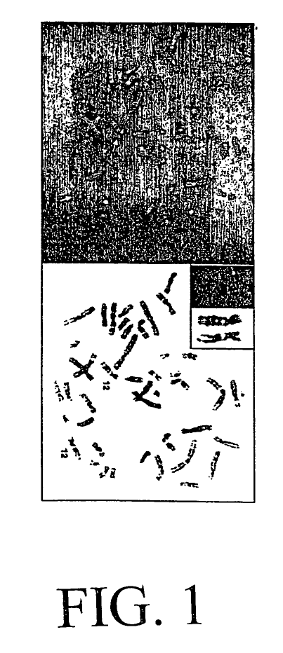

The usefulness of genomic-based studies for the discovery and investigation of genes expressed in proliferating erythroid cells has been previously demonstrated¹⁹. To search for genes that encode molecules expressed on circulating erythrocytes like the Dombrock molecule, it was rationalized that a genomic analysis of more differentiated erythroid cells may be required. For this purpose, peripheral blood mononuclear cells from blood donors were cultured in the presence of erythropoietin, and flow cytometry was used to isolate highly purified populations of erythroid cells undergoing terminal differentiation after 12 days in culture¹⁶. A unidirectional cDNA library was constructed from sorted cells and a database comprising about 5000 ESTs was generated. Two criteria were applied to screen the database for Dombrock candidates: (i) the candidate gene should localize to chromosome 12p in the region previously linked to Dombrock polymorphism and (ii) a signal peptide required for surface localization of GPI-proteins should be encoded in the 5′ sequence. Based upon the examination of individual BLAST homology comparisons (http://www.ncbi.nlm.nih.gov/blast/) for each sequence, one candidate EST from the library conformed to these two criteria. The clone shared significant homology with a recently sequenced BAC clone (GenBank accession number AC007655) derived from chromosome 12p. Notably, that BAC sequence also carries a marker (D12S932) in the genomic region previously linked to the Dombrock blood group¹⁴. FISH analysis was performed to confirm the location of the BAC clone in this region of chromosome 12 (FIG. 1.).

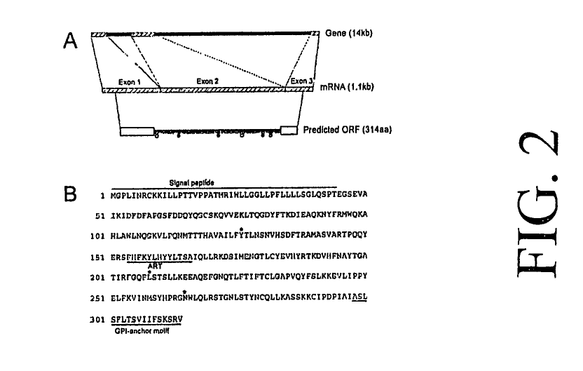

Full-length sequencing of the candidate cDNA clone (referred to here as DOK1) revealed a 1.1 kb cDNA with an open reading frame (ORF) of 945 bp (FIG. 2). The gene is comprised of 3 exons spanning 14 kb. The DOK1 ORF encodes a protein of 314 amino acid residues with the characteristic N-terminal signal peptide and C-terminal GPI-anchor attachment motif. Several possible sites for N-linked glycosylation and N-myristoylation are present within the coding region. An ADP-ribosyltransferase (ART) motif was predicted within exon 2 that is highly homologous to the first exon of the ADP-ribosyltransferase gene family member ART4²⁰. Alignment studies suggest that DOK1 and ART4 share two exons transcribed from chromosome 12p.

DOK1 Expression Pattern

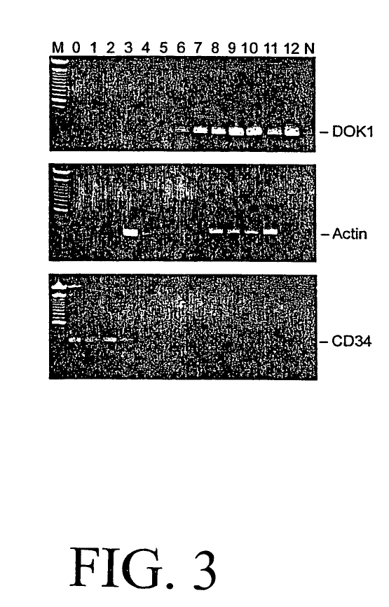

To examine expression of DOK1 in erythroid cells, RT-PCR assay was performed using RNA from mobilized CD34+ peripheral blood cells cultured for 2 weeks in the presence of erythropoietin (EPO). During that period, the CD34+ cells differentiate into glycophorin A expressing erythroid precursors. Intron-spanning PCR primers were chosen to detect expression of the DOK1, CD34, and PBGD genes. As shown in FIG. 3, DOK1 expression was not detected before day 4 in culture. The relative signal became stronger over the next week and correlated with the loss of CD34 expression. By day 12, the DOK1 signal was comparable to that of PBGD, while CD34 expression was no longer seen.

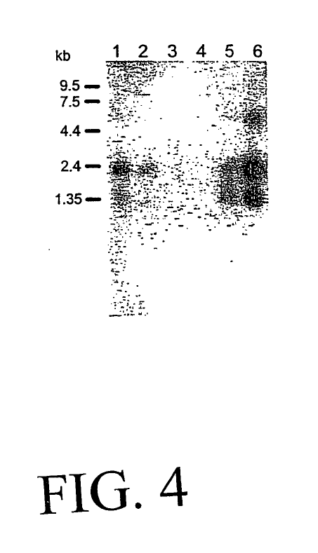

Human Immune System Multiple Tissue Northern (MTN™) Blot II (Clontech, Palo Alto, Calif.) was used to assess the expression of DOK1 in hematologic tissues. The DOK1 probe hybridized to three bands having sizes of about 1, 2, and 7 kb. Bands were apparent in spleen, lymph node, bone marrow and fetal liver (FIG. 4). No hybridization was observed in thymus or peripheral blood leukocytes. The strongest signal was present in the lane containing RNA from fetal liver, consistent with erythroid cell production in that organ. A homology search of DOK1 against EST databases also suggests the DOK1 gene is primarily expressed in the fetal liver and spleen.

Serologic Testing of DOK1 as Molecular Carrier of Dombrock Antigens

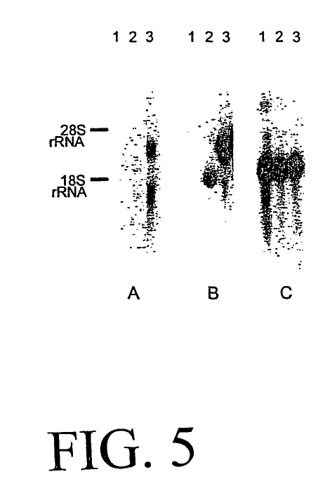

Several cell types (K562, peripheral blood leukocytes, MEG, Jurkat, HEL) were further screened for the expression of DOK1 by RT-PCR. Only HEL erythroleukemic cells demonstrated expression of DOK1. Since the K562 erythroleukemic cell line had no detectable expression of DOK1, those cells were chosen for serologic testing. To create stable transfectants, the DOK1 open reading frame was subcloned into a commercially available vector pIRES2EGFP (pI2E; control vector) to create the plasmid pDOK1IRES2EGFP pDI2E; expressing DOK1). Identification of stable K562 transfection pools was achieved by G418 selection for at least 2 weeks and green fluorescent protein (GFP) expression. Northern blotting of untransfected K562 cells and K562 cells transfected either with pI2E or pDI2E confirmed expression of DOK1 only in pDI2E transfected cells (FIG. 5).

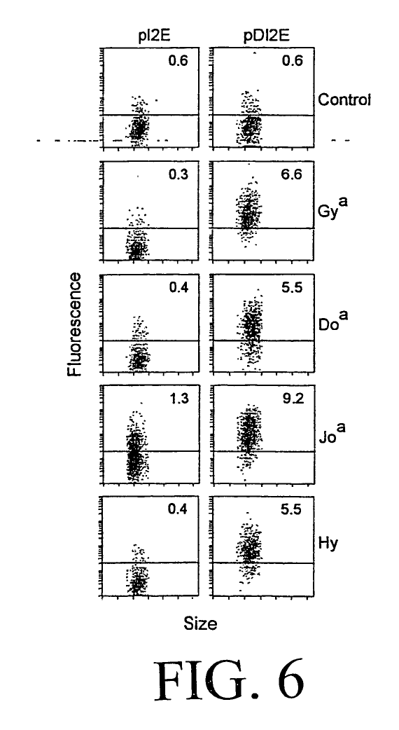

Cells expressing GFP among the pDI2E and pI2E transfected populations were then assayed for Dombrock serum antibody binding. Sera negative for Dombrock agglutinating antibodies were used as controls. Dombrock serum antibody binding to the DOK1 transfected cells (pDI2E) resulted in a shift in mean fluorescence to levels greater than two standard deviations above the control cells (pI2E transfected). As shown in FIG. 6, Dombrock specific serum binding was demonstrated for all of the antigens tested. Overall, 14 sera representing 4 of 5 antigens (Do^{b} antisera was not available) were assayed. As shown in Table 1, 9 of 14 demonstrated specific, high-level binding to the DOK1 expressing cells. Only 1 of 5 anti-Hy sera demonstrated specific binding to the DOK1 expressing population.

DOK1 Encodes a GPI-Protein

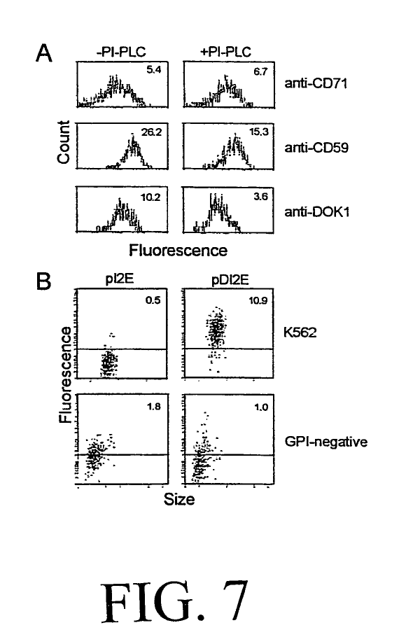

Since the Dombrock molecule is reportedly GPI-anchored, tests were performed to determine whether DOK1 encodes a GPI-protein. Flow cytometry was used to compare the level of anti-Dombrock sera binding to pDI2E expressing cells before and after treatment with phosphatidylinositol phospholipase C (PI-PLC). Assays of CD71 (transmembrane protein) and CD59 (GPI-protein) cleavage were used as negative and positive controls, respectively. PI-PLC mediated cleavage of GPI-proteins was measured after staining with anti-CD71, anti-CD59 or anti-Dombrock antibodies (FIG. 7, A). PI-PLC action produced no significant changes in mean fluorescence in cells stained with anti-CD71 antibodies (mean fluorescence 5.4 versus 6.7). In contrast, the level of CD59 on the surface membrane was significantly reduced (mean fluorescence 26.2 versus 15.3). Staining with Dombrock specific antisera revealed a pattern similar to that of anti-CD59 stained cells (mean fluorescence 10.2 versus 3.6). This 3-fold reduction of mean fluorescence is consistent with findings that DOK1 is anchored to the plasma membrane via glycosylphosphatidylinositol.

The pDI2E plasmid was also introduced into K562 cells unable to express GPI-proteins on their plasma membranes (GPI-negative cells) to independently confirm that DOK1 is an GPI-anchored protein²⁰. Parental K562 and GPI-negative K562 cells were transfected with pI2E and pDI2E. Expression of DOK1 on the surface of the cells was analyzed by flow cytometry following the staining with anti-Dombrock serum (FIG. 7, B). The parental K562 cells transfected with pDI2E exhibited strong binding to anti-DOK1 antibodies while pI2E transfected K562 cells did not. The GPI-negative cells transfected either with pDI2E or pI2E failed to show any specific binding of the anti-Dombrock serum, while GFP expression from internal ribosome entry site (IRES) of pI2E or pDI2E was observed in both cases. Hence, DOK1 demonstrated PI-PLC sensitivity on K562 cells and a lack of surface expression among GPI-negative cells. Both properties are consistent with the computer prediction that DOK1 is a GPI-protein.

Single Nucleotide DOK1 Polymorphisms

In order to determine whether genetic polymorphisms correlate with blood group antigenicity, genomic DNA or reticulocyte RNA was isolated from eight blood donors of defined serology (four Do(a+b−) versus four Do(a−b+)) and looked for donor-specific differences in the DOK1. The DOK1 coding regions from the samples were sequenced and aligned. The alignments revealed three single nucleotide polymorphic sites (SNPs) within the coding region of DOK1 (FIG. 2.). While two SNPs did not alter the predicted amino acid primary structure (Y126, L208), the third predicts a mutation in the protein sequence N265D). Notably, the N265D mutation falls within an RGD adhesion motif of the molecule. All three SNPs were consistent among the eight donors with N265 found in the four Do(a+b−) and D265 present in the four Do(a−b+) samples, respectively.

Detecting Anti-Dombrock (a) or Anti-Dombrock (b) Antibodies.

Brief Discussion of the test: The systematic screening of patient and/or donor serums enables unexpected antibodies to be detected. The subsequent testing of a serum found to contain unexpected antibodies, using a panel of typed red blood cell suspensions, enables the specificity of the antibody to be determined, in order to select donor blood negative for the appropriate antigen(s) for transfusion, or to identify serums that may be suitable for antiserum preparation.

Principle of the test: Serums found to contain unexpected antibodies are tested by appropriate test procedures against a panel of selected red blood cells suspensions. The pattern of reactions obtained with the selected panel of cells is compared with the known antigen makeup of the panel, thereby enabling the identity of the unexpected antibody to be determined.

Reagent description: The kit comprises twenty human group O red blood cell suspensions, selected to provide a greater range of phenotype combinations than is possible with a panel of ten, eleven or even of sixteen cell suspensions, thereby being especially useful in the identification and/for confirmation of antibody mixtures. The individual suspensions are derived from the blood of a single donor, washed to remove blood group antibodies, then resuspended to a concentration of 3-4% in a modified Alsever's Solution containing Neomycin Sulfate (0.0068 g/% reactivity) and Chloramphenicol (0.033 g/%) as preservative agents, as well as Inosine 0.4 g/% to preserve carbohydrate metabolism. The suspending medium does not inhibit complement-mediated hemolysis. Antigens for which the cells have been typed are noted on the PanelSheet™ enclosed with each product lot. These products are prepared under U.S. government license and are intended for in-vitro diagnostic use only. Reactivity of Reagent Red blood Cells may diminish during the dating period. Store at 1° to 8° C. when not in use. Do not freeze. Exercise care to protect from contamination. Do not use if markedly homolyzed.

Specimen collection and preparation: No special preparation of the patient is required prior to specimen collection. Blood should be drawn by an aseptic technique and the serum should be tested as soon as possible. If delay in testing should occur, the specimen must be stored at 1° to 8° C. In the case of potential blood transfusion recipients, the specimens should be stored for no longer than is permitted by the relevant regulatory agencies. Antibodies dependent for their detection upon the binding of complement may not be detected if aged serum or plasma from an anticoagulated sample are used for antibody detection tests.

Procedure:

Reagents Supplied: Reagent Red Blood Cells for antibody Identification: Test lubes (75×12 mm or 75×10 mm), pipettes, glass slides, physiologic saline, 37° C. waterbath or incubator, timer and centrifuge, as well as an optical aid such as a hand lens, a concave mirror or a microscope.

Additional Reagents Required or Recommended: An antibody potentiator such as 22% or 30% Bovine Albumin, or a low-ionic-strength additive solution. If desired, a low-ionic-strength solution (LISS) may be used to replace Alsever's Solution as the cell suspending medium. (Note, however, that Reagent Red Blood Cells must be stored in LISS. If using this technique, prepare only sufficient red blood suspension for a single day's testing. Any unused cell suspension must be discarded at the end of the day.)

Test Procedure:

The test procedure detailed below is one that is in common use. It may be varied according to the requirements and experience of the particular laboratory.
1. For each serum, label an appropriate number of mall test tubes (75×12 mm 75×10 mm), one tube for each cell suspension to be tested. If the serum is tested against the whole panel, twenty tubes are required. A further tube should be labeled for an auto control test. When attempting to identify an unexpected antibody, it is usually considered sensible to test, together with the panel, at least one cell suspension known to have given a positive reaction with the serum being tested. For example, a reagent red blood cell suspension with which a positive screening test was obtained, or a suspension prepared from the donor whose cells showed incompatibility. If tests are being carried out to identify the unexpected antibody known to be reactive in a saline test system at room temperature, a second set of labeled tubes will be required for the saline tests.
2. Place 2-3 drops of the serum to be tested in each tube (or such lesser volume as may be specified for the low-ionic-strength method in use).
3. Add one drop of the appropriate cell suspension to each appropriate tube. If using a low-ionic-strength test procedure, the cell suspensions should be prepared accordance with the directions for use supplied with the particular product. Note: If using 22% or 30% Bovine Albumin as a poteniator of agglutination, a reactivity of weak antibodies may be enhanced by washing the cells ore time physiologic saline, decanting the saline completely and using the “dry button” cells for the test. If this procedure is followed, Step 2 would be the preparation of the cells in the tube in which the test is to be carried out. Addition of the serum to be tested would their become Step 3.
4. Add 2 or 3 drops of 22% or 30% Bovine Albumin to each tube in which it is intended that Bovine Albumin will be used as a potentiator. This step is omitted if the cells have been suspended in low-ionic-strength solution (LISS), and in the case of tests to be performed in a saline room temperature test system. A low ionic-strength additive solution may be used in place of albumin, if desired. See directions insert for the chose low-ionic-strength additive.
5. Centrifuge all tubes for a time appropriate to the calibration of the centrifuge. Note: The centrifugal force applied to mixtures of serum and cells should be the minimum needed to yield a compact “button” of red cells and a clear supernate. Excessive centrifugation leads to difficulty in resuspending the cell button, which inadequate centrifugation may yield agglutinates that are too readily dispersed. Each laboratory should calibrate its own centrifuges to determine optimal times and speeds of centrifugation for different test systems. As a guide, 1 minute at 1,000 rpm (rcf 100-125) is usually adequtate for both saline and albumin tests. A 3,400 rpm (rcf 900-1,000) saline tests usually require 15 seconds, while albumin tests require 30 seconds due to the greater viscosity of bovine albumin.
6. Examine for hemolysis and record if present. Note: Assume the absence of bacterial or chemical contamination, hemolysis may indicate an antigen/antibody reaction. Most often, antibodies capable of producing hemolysis have specificity in the ABO, P, Lewis, Kidd or Vel blood group systems.
7. Resuspend the cells by gentle shaking and examine macroscopically for agglutination. Record the results appropriately? (e.g., “immediate-spin saline,” etc.)
8. Incubate the tubes at 37° C.±1° C. according to the manufacturer's directions fort eh potentiator being used. If no potentiator or addictive is used, incubation for 30-60 minutes at at 37° C.±1° C. is recommended. (If the serum is also being tested by a saline room temperature procedure, incubate the saline tubes at at 23° C.±3° C. for 15-60 minutes.)
9. Centrifuge all tubes as noted under step 5.
10. Repeat steps 6 and 7 and record results appropriately.
11. If a saline room temperature test was being done, discard those tubes.
12. Wash the cells in the remaining tubes at least 3 times with the tubes full of saline being careful to decant the saline between washes and to resuspend the cells thoroughly when adding saline for the next wash.
13. Decant the saline completely following the last wash.
14. Add 1 or 2 drops of Gamma Anti-Human Globulin to each “dry button” of cells.
15. Mix well and centrifuge for:
   (a) 1 minute at 1,000 rpm (rcf 100-125)
   or (b) 15 seconds at 3,400 rpm (rcf 900-1,000)
   or (c) a time appropriate to the calibration of the centrifuge.
16. Resupsend the cells by gentle shaking.
17. Examine for agglutination and record test results. Note: A variety of methods may be used to examine for agglutination, such as the unaided eye, a hand lens, a concave mirror or a microscope. Since each lot of Gamma Anti-Human Globulin is tested and found to yield specific results when observed microscopically, the method used to determine agglutination depends on the training and experience of the individual worker.
   Stability of the Final Reaction: The washing phases of the antiglobulin test should be carried out without interruption, and final test results should be interpreted immediately upon completion the test.
   CONTROLS: Proper controls are essential in the performance of all laboratory procedures.
1. Strength of a given red blood cell antigen, over the dating period, may be monitored by testing the cell suspension periodically for relative antigen strength with a known antibody.
2. All negative antiglobulin tests should be confirmed by adding IgG-sensitized cells, such as Gamma Coombs Control Cells, and then repeating centrifugation and reading. A positive test result at this point will confirm that active antiglobulin was added to the test system and was present when the original antiglobulin test was interpreted as negative.
3. Though not an important feature in the initial antibody screening, an auto control test is strongly recommended for antibody identification tests. This consists of testing the serum against the person's own red cells, in parallel with the Reagent Red Blood Cells.
   INTERPRETATION OF TEST RESULTS: Agglutination or hemolysis of any panel cell, suspension in the immediate-spin or incubation phases of testing, or agglutination occurring at the antiglobulin phase, constitutes a positive test result and indicates the presence of one or more antibodies directed at antigens present on the particular cells. The absence of hemolysis or agglutination constitutes a negative test result and indicates the absence of detectable antibodies to antigens present on the cells. If a pattern of positive and negative reactions is observed with different test cells, antibody identification is simplified by eliminating antibodies specific or antigens present on any non-reactive cells. Comparison of the pattern of positive and negative reactions with the known antigen makeup of the reagent red blood cells will enable the specificity of the antibody or antibodies to be determined or will lead to absorption and/or ellution tests needed to separate the component specificities of antibody mixtures. If an auto control test is performed and gives a positive result, the test serum contains an autoantibody. In the case of a recently transfused patient, a positive auto control test may indicate the presence of an alloantibody directed at an antigen present on surviving donor calls. In such cases mixed-field agglutination reaction may be seen.

Throughout this application, various publications are referenced. The disclosures of these publications in their entireties are hereby incorporated by reference into this application in order to more fully describe the state of the art to which this invention pertains.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

### REFERENCES

1. Garratty, G. Dzik W H, Issitt P D, Lublin D M, Reid M, Zelinski T. Terminology for blood group antigens and genes: Historical origins and guidelines in the new millennium. Transfusion. 2000;40:477-489.
2. Daniels, G. Functional aspects of red cell antigens. Blood Rev. 1999;13:14-35.
3. Hadley T J, Peiper S C. From malaria to chemokine receptor: the emerging physiologic role of the Duffy blood group antigen. Blood. 1997;89:3077-3091.
4. Zimmerman P A, Woolley I, Masinde G L, et al. Emergence of FY*A(null) in a Plasmodium vivax-endemic region of Papua New Guinea. Proc Natl Acad Sci USA. 1999; 96:13973-13977.
5. Mourant E, Kopec A C, Domaniewska-Sobczak K. Blood Groups and Diseases: A study of associations of diseases with blood groups and other polymorphisms. Oxford, Oxford University Press; 1978.
6. Swanson J, Polesky HF, Tippett P, Sanger R. A “New” Blood Group Antigen, Do^{a}, Nature. 1965;206:313.
7. Molthan L, Crawford M N, Tippett P. Enlargement of the Dombrock blood group system: the finding of anti-Do^{b}. Vox Sang. 1973;24:382-384.
8. Daniels G. Human Blood Groups. Cambridge, Blackwell Science; 1995.
9. Banks J A, Hemming N, Poole J. Evidence that the Gya, Hy and Joa antigens belong to the Dombrock blood group system. Vox Sang 1995;68(3):177-82.
10. Halverson G, Shanahan E, Santiago I, et al. The first reported case of anti-Dob causing an acute hemolytic transfusion reaction. Vox Sang. 1994;66: 206-209.
11. Shirey, R S, Boyd J S, King K E, Caturegli P P, Montgomery W M Jr, Ness P M. Assessment of the clinical significance of anti-Dob. Transfusion. 1998;38:1026-1029.
12. Strupp A, Cash, K, Uehlinger J. Difficulties in identifying antibodies in the Dombrock blood group system in multiply alloimmuunized patients. Transfusion. 1998;38:1022-1025.
13. Lewis M, Kaita, H, Giblett, E R, Anderson J E. Genetic linkage analysis of the Dombrock (Do) blood group locus. Cytogenet. Cell. Genet. 1978;22:313-318.
14. Eiberg H, Mohr J. Dombrock blood group (DO): assignment to chromosome 12p. Hum. Genet. 1996;98:518-521.
15. Telen M J, Rosse W F, Parker C J, Moulds M K, Moulds J J. Evidence that several high-frequency human blood group antigens reside on phosphatidylinositol-linked erythrocyte membrane proteins. Blood. 1990; 75:1404-1407.
16. Miller J L, Njoroge J M, Gubin A N, Rodgers G P. Prospective identification of erythroid elements in cultured peripheral blood. Exp. Hematol. 1999;27:624-629.
17. Pack S D, Zbar B, Pak E, et al. Constitutional von Hippel-Lindau (VBL) gene deletions detected in VHL families by fluorescence in situ hybridization Cancer Res. 1999;59:5560-4.
18. Duro G, Barbieri R, Ribaudo M R, Feo S, Izzo V. A downward capillary blotting procedure from hundred base pairs to hundred kilobases nucleic acids. Anal. Biochem. 1995;225:360-362.
19. Gubin A N, Njoroge J M, Bouffard G G, Miller J L. Gene expression in proliferating human erythroid cells. Genomics. 1999;59:168-177.
20. Koch-Nolte F, Haag F, Braren R, et al. Two novel human members of an emerging mammalian gene family related to mono-ADP-ribosylating bacterial toxins. Genomics 1997;39:370-376.
21. Hirose S, Mohney R P, Mutka S C, et al. Derivation and characterization of glycoinositol-phospholipid anchor-defective human K562 cell clones. J. Biol. Chem. 1992;267:5272-5278.
22. Reid M E, McManus K, Zelinski T. Chromosome location of genes encoding human blood groups. Transfus. Med. Rev. 1998;12:151-161.
23. Cartron J P. A molecular approach to the structure, polymorphism and function of blood groups. Transfus. Clin. Biol. 1996;3:181-210.
24. Telen M J, Bolk T A. Human red cell antigens. IV. The abnormal sialoglycoprotein of Gerbich-negative red cells. Transfusion. 1987;27:309-314.
25. Haag F, Koch-Nolte, F. ADP-Ribosylation in Animal Tissues Structure, Function, and Biology of Mono (ADP-ribosyl) Transferases and Related Enzymes. New York and London, Plenum Press;1997.
26. Okazaki I J, Moss J. Characterization of glycosylphosphatidylinositiol-anchored, secreted, and intracellular vertebrate mono-ADP-ribosyltransferases. Annu. Rev. Nutr. 1999;19:485-509.
27. Greiner D L, Handler E S, Nakano K, Mordes J P, Rossini A A. Absence of the RT-6 T cell subset in diabetes-prone BB/W rats. J. Immunol. 1986;136:148-151.
28. Nishimura J, Murakami Y, Kinoshita T. Paroxysmal nocturnal leemoglobinuria: An acquired genetic disease. Am. J. Hematol. 1999;62:175-182.
29. Spring F A, Reid M E. Evidence that the human blood group antigens Gya and Hy are carried on a novel glycosylphosphatidylinositol-linked erythrocyte membrane glycoprotein. Vox Sang. 1991;60:53-59.
30. Scarborough R M. Structure-activity relationships of beta-amino acid-containing integrin antagonists. Curr Med Chem 1999; 10:971-81.
31. Zolkiewska A, Moss J. Integrin alpha 7 as substrate for a glycosylphosphatidylinositol-anchored ADP-ribosyltransferase on the surface of skeletal muscle cells. J. Biol. Chem. 1993;268:25273-25276.
32. Ruiz-Jarabo C M, Sevilla N, Davila M, Gomez-Mariano G, Baranowski A, Domingo E. Antigenic properties and population stability of a foot-and-mouth disease virus with an altered Arg-Gly-Asp receptor-recognition motif. J. Gen. Virol. 1999;80: 1899-1909.

33. Robson K J, Hall J R, Jennings M W, et al. A highly conserved amino-acid sequence in thrombospondin, properdin and in proteins from sporozoites and blood stages of a human malaria parasite. Nature 1988;335:79-82.

TABLE 1

| Sera | # Tested | # Positive |
|---|---|---|
| Control | 3 | 0 |
| Gy^{a} | 4 | 4 |
| Do^{a} | 1 | 1 |
| Jo^{a} | 4 | 3 |
| Hy | 5 | 1 |

Table 1. Serologic assessment of DOK1. Antisera for the Hy, Gy^{a}, Jo^{a}, and Do^{a} antigens were incubated with cells transfected with pDI2E then secondarily stained (see methods). Serum from donors without detectable anti-Dombrock agglutination activity were used as negative controls (Control). The serum assay was scored positive if the mean fluorescence of the pDI2E transfected cells fluoresced at levels greater than two standard deviations above the controls. All tests were performed in duplicate or triplicate.

1. An isolated peptide having the amino acid sequence of SEQ ID NO: 2, wherein the peptide is a Do(a) antigen.

2. An isolated antigenic fragment of the peptide of claim 1, wherein the fragment comprises asparagine at position 265.

3. An isolated nucleic acid, encoding the peptide of claim 1.

4. The nucleic acid of claim 3, wherein the nucleic acid comprises the nucleotide sequence of SEQ ID NO: 1.

5. An isolated fragment of the nucleic acid of claim 3, wherein the fragment encodes an antigenic fragment of SEQ ID NO:2.

6. An isolated peptide having the amino acid sequence of SEQ ID NO:4, wherein the peptide is a Do(b) antigen.

7. An isolated fragment of the peptide of claim 6, wherein the fragment comprises aspartic acid at position 265.

8. An isolated nucleic acid, encoding the peptide of claim 6.

9. The nucleic acid of claim 8, wherein the nucleic acid comprises the nucleotide sequence of SEQ ID NO. 3

10. An isolated fragment of the nucleic acid of claim 8, wherein the fragment encodes an antigenic fragment of SEQ ID NO: 4.

11. An isolated fragment of the nucleic acid of claim 4, comprising at least 10 nucleotides, wherein the fragment comprises cytosine at position 477 of SEQ ID NO: 1.

12. An isolated fragment of the nucleic acid of claim 4, comprising at least 10 nucleotides, wherein the fragment comprises thymine at position 723 of SEQ ID NO: 1.

13. An isolated fragment of the nucleic acid of claim 4, comprising at least 10 nucleotides, wherein the fragment comprises adenine at position 892 of SEQ ID NO: 1.

14. An isolated fragment of the nucleic acid of claim 9, comprising at least 10 nucleotides, wherein the fragment comprises thymine at position 477 of SEQ ID NO: 3.

15. An isolated fragment of the nucleic acid of claim 9, comprising at least 10 nucleotides, wherein the fragment comprises cytosine at position 723 of SEQ ID NO: 3.

16. An isolated fragment of the nucleic acid of claim 9, comprising at least 10 nucleotides, wherein the fragment comprises guanine at position 892 of SEQ ID NO: 3.

17. An antibody that specifically binds the peptide of claim 1.

18. The antibody of claim 17, wherein the antibody is monoclonal.

19. An antibody that specifically binds the antigenic fragment of claim 2.

20. The antibody of claim 19, wherein the antibody is monoclonal.

21. An antibody that specifically binds the peptide of claim 6.

22. The antibody of claim 21, wherein the antibody is monoclonal.

23. An antibody that specifically binds the antigenic fragment of claim 7.

24. The antibody of claim 23, wherein the antibody is monoclonal.

25. A kit for detecting anti-Dombrock A antibodies, comprising the peptide of claim 1.

26. A kit for detecting anti-Dombrock A antibodies comprising the fragment of claim 2.

27. A kit for detecting anti-Dombrock B antibodies comprising the peptide of claim 6.

28. A kit for detecting anti-Dombrock B antibodies comprising the fragment of claim 7.

29. A kit for detecting a Dombrock A antigen comprising the antibody of claim 18.

30. A kit for detecting a Dombrock A antigen comprising the antibody of claim 20.

31. A kit for detecting a Dombrock B antigen comprising the antibody of claim 22.

32. A kit for detecting a Dombrock B antigen comprising the antibody of claim 24.

33. A kit for detecting a Dombrock A antigen comprising the nucleic acid of claim 3.

34. A kit for detecting a Dombrock A antigen comprising the nucleic acid of claim 4.

35. A kit for detecting a Dombrock A antigen comprising the nucleic acid of claim 5.

36. A kit for detecting a Dombrock B antigen comprising the nucleic acid of claim 8.

37. A kit for detecting a Dombrock B antigen comprising the nucleic acid of claim 9.

38. A kit for detecting a Dombrock B antigen comprising the nucleic acid of claim 10.

39. A kit for detecting a Dombrock A antigen comprising the nucleic acid of claim 11.

40. A kit for detecting a Dombrock A antigen comprising the nucleic acid of claim 12.

41. A kit for detecting a Dombrock A antigen comprising the nucleic acid of claim 13.

42. A kit for detecting a Dombrock B antigen comprising the nucleic acid of claim 14.

43. A kit for detecting a Dombrock B antigen comprising the nucleic acid of claim 15.

44. A kit for detecting a Dombrock B antigen comprising the nucleic acid of claim 16.

45. A kit comprising a microchip array comprising a nucleic acid selected from the group consisting of an isolated nucleic acid encoding the peptide having an amino acid sequence of SEQ ID NO:2; the nucleic acid comprising the nucleotide sequence of SEQ ID NO:1; an isolated fragment of a nucleic acid that encodes the peptide having an amino acid sequence of SEQ ID NO:2; an isolated fragment of the nucleotide sequence of SEQ ID NO:1, comprising at least 10 nucleotides, wherein the fragment comprises cytosine at position 477 of SEQ ID NO: 1; an isolated fragment of the nucleotide sequence of SEQ ID NO:1, comprising at least 10 nucleotides, wherein the fragment comprises thymine at position 723 of SEQ ID NO: 1; an isolated fragment of the nucleotide sequence of SEQ ID NO: 1, comprising at least 10 nucleotides, wherein the fragment comprises adenine at position 892 of SEQ ID NO:1; an isolated nucleic acid encoding the peptide having an amino acid sequence of SEQ ID NO:4; the nucleic acid comprising the nucleotide sequence of SEQ ID NO:3; an isolated fragment of a nucleic acid that encodes the peptide having an amino acid sequence of SEQ ID NO:4; an isolated fragment of the nucleotide sequence of SEQ ID NO:3, comprising at least 10 nucleotides, wherein the fragment comprises thymine at position 477 of SEQ ID NO:3; an isolated fragment of the nucleotide sequence of SEQ ID NO:3, comprising at least 10 nucleotides, wherein the fragment comprises cytosine at position 723 of SEQ ID NO:3; and an isolated fragment of the nucleotide sequence of SEQ ID NO:3, comprising at least 10 nucleotides, wherein the fragment comprises guanine at position 892 of SEQ ID NO:3.

46. A kit comprising red blood cells having a Dombrock A antigen.

47. A kit comprising red blood cells having a Dombrock B antigen.

48. A method of detecting in a sample an antibody directed to a Dombrock carrier molecule, comprising:
a) contacting red blood cells having a Dombrock carrier molecule with the sample; and
b) detecting agglutination of the red blood cells, whereby agglutination of the red blood cells indicates the presence in the sample of an antibody to a Dombrock carrier molecule.

49. The method of claim 48, wherein the antibody being detected is anti-Dombrock A, and the Dombrock carrier molecule contains the Dombrock A antigen.

50. The method of claim 48, wherein the antibody being detected is anti-Dombrock B, and the Dombrock carrier molecule contains the Dombrock B antigen.

51. A method of detecting in a sample an antibody directed to a Dombrock carrier molecule, comprising:
a) contacting a purified Dombrock carrier molecule with the sample; and
b) detecting an antigen/antibody complex, whereby detection of the antigen/antibody complex indicates the presence of the antibody directed to a Dombrock carrier molecule.

52. The method of claim 51, wherein the antibody being detected is anti-Dombrock A, and the antigen is the peptide of claim 1.

53. The method of claim 51, wherein the antibody being detected is anti-Dombrock B, and the antigen is the peptide of claim 6.

54. A method of detecting a Dombrock carrier molecule on red blood cells, comprising:
a) contacting the red blood cells with an antibody directed to the Dombrock carrier molecule; and
b) detecting agglutination of the red blood cells, whereby agglutination of the red blood cells indicates the presence of the Dombrock carrier molecule.

55. The method of claim 54, wherein the Dombrock carrier molecule comprises Dombrock A and the antibody is the antibody directed to Dombrock A.

56. The method of claim 54, wherein the Dombrock carrier molecule comprises Dombrock B and the antibody is the antibody directed to Dombrock B.

57. A method of detecting in a sample a Dombrock carrier molecule, comprising:
a) contacting the sample with an antibody directed to a Dombrock carrier molecule; and
b) detecting a carrier molecule/antibody complex, whereby detection of the carrier molecule/antibody complex indicates the presence of the Dombrock carrier molecule in the sample.

58. The method of claim 57, wherein the Dombrock carrier molecule is has a Dombrock A antigen.

59. The method of claim 57, wherein the Dombrock carrier molecule has a Dombrock B antigen.

60. A method of detecting a subject having a Dombrock carrier molecule, comprising:
a) performing amplification of a nucleic acid of the subject by contacting a nucleic acid from a cell of the subject with a primer having a nucleotide sequence specific for a nucleic acid encoding a Dombrock carrier molecule; and
b) detecting an amplification product from step (a), whereby the detection of an amplification product identifies the subject as having a Dombrock carrier molecule.

61. The method of claim 60, wherein the Dombrock carrier molecule has a Dombrock A antigen.

62. The method of claim 60, wherein the Dombrock carrier molecule has a Dombrock B antigen.

63. A method of detecting in a sample, a cell that expresses a Dombrock A antigen, comprising detecting in the sample a nucleic acid that encodes a Dombrock A antigen.

64. A method of detecting in a sample, a cell that expresses a Dombrock B antigen, comprising detecting in the sample a nucleic acid that encodes a Dombrock B antigen.
